(19) United States
(12) Patent Application Publication
(10) Pub. No.: US 2025/0344978 A1
(43) Pub. Date: Nov. 13, 2025

(54) HEART TISSUE IDENTIFICATION IN THE CONTEXT OF ATRIAL FIBRILLATION

(71) Applicant: AUCKLAND UNISERVICES LIMITED, Auckland (NZ)

(72) Inventors: Bruce SMAILL, Auckland (NZ); Shu MENG, Auckland (NZ); David BUDGETT, Auckland (NZ)

(21) Appl. No.: 18/860,357

(22) PCT Filed: Apr. 27, 2023
(86) PCT No.: PCT/IB2023/054396

§ 371 Date: Oct. 25, 2024

Related U.S. Application Data
(60) Provisional application No. 63/336,265, filed on Apr. 28, 2022.

(51) Int. Cl. A61B 5/287; A61B 5/361; A61B 18/00; A61B 18/14

(52) U.S. Cl. CPC A61B 5/287; A61B 5/361; A61B 18/1492; A61B 2018/00351; A61B 2018/00577; A61B 2018/00839

(57) ABSTRACT

A method, including obtaining heart phase data for a plurality of activation cycles of a living human afflicted with atrial fibrillation and analyzing the heart phase data to identify specific heart tissue locations where there are repeated and consistent temporal discrepancies of electrical activation relative to other tissue locations, wherein the action of analyzing the heart phase data includes executing a statistical analysis on the heart phase data.

### CROSS-REFERENCE TO RELATED APPLICATIONS

This application claims priority to U.S. Provisional Application No. 63/336,265, entitled HEART TISSUE IDENTIFICATION IN THE CONTEXT OF ATRIAL FIBRILLATION, filed on Apr. 28, 2022, naming Bruce SMAILL as an inventor, the entire contents of that application being incorporated herein by reference in its entirety.

### BACKGROUND

Atrial fibrillation (afib) is an irregular and often very rapid heart rhythm (arrhythmia). Atrial fibrillation increases the risk of stroke, heart failure and other heart-related complications.

During atrial fibrillation, the heart's upper chambers (the atria) beat chaotically and irregularly—out of sync with the lower chambers (the ventricles) of the heart. For many people, atrial fibrillation may have no symptoms. However, A-fib may cause a fast, pounding heartbeat (palpitations), shortness of breath or weakness.

Episodes of atrial fibrillation may come and go, or they may be persistent. Atrial fibrillation can be a serious medical condition that requires proper treatment to prevent stroke.

Treatment for atrial fibrillation may include medications, therapy to reset the heart rhythm, and catheter procedures to block faulty heart signals.

The typical heart has four chambers—two upper chambers (atria) and two lower chambers (ventricles). Within the upper right chamber of the heart (right atrium) is a group of cells called the sinus node. The sinus node is the heart's natural pacemaker. It produces the signal that starts each heartbeat.

In a regular heart rhythm, the signal travels from the sinus node through the two upper heart chambers (atria), the signal passes through a pathway between the upper and lower chambers called the atrioventricular (AV) node and the movement of the signal causes your heart to squeeze (contract), sending blood to the heart and body.

In atrial fibrillation, the signals in the upper chambers of the heart are chaotic. As a result, the upper chambers shake (quiver). The AV node is then bombarded with signals trying to get through to the lower heart chambers (ventricles). This causes a fast and irregular heart rhythm.

The heart rate in atrial fibrillation may range from 100 to 170 or 180 or more beats per minute. In contrast, the normal range for a heart rate is 60 to 90ish beats a minute.

Preventative concepts are often limited to choosing a healthy lifestyle believed to reduce the risk of heart disease and may prevent atrial fibrillation, such as managing stress, as intense stress and anger can cause heart rhythm problems.

Also, there are medicines to control the heart's rhythm and rate, blood-thinning medicine to prevent blood clots from forming and reduce stroke risk and medicine and healthy lifestyle changes to manage atrial fibrillation risk factors. More specifically, medications include beta blockers, blood thinners, calcium channel blockers, and heart rhythm medicines.

### SUMMARY

In an embodiment, there is a method, comprising obtaining heart phase data for a plurality of activation cycles of a living human afflicted with atrial fibrillation and analyzing the heart phase data to identify specific heart tissue locations where there are repeated and consistent temporal discrepancies of electrical activation relative to other tissue locations.

In an embodiment, there is a method, comprising developing a time-varying electrical potential map of a surface of a cavity of a beating heart, developing a time-varying phase map of the surface of the cavity based on the developed time-varying electrical potential map, and identifying repeating phase signatures for respective locations on the surface of the atrial cavity from the time-varying phase map that repeat in a statistically aberrant manner relative to other phase signatures at other respective locations.

In an embodiment, there is a method, comprising developing data including at least X spatial locations and at least Y respective phase gradients for the respective spatial locations of the X spatial locations, statistically analyzing the developed data, identifying locations of the respective locations that are indicative of tissue influencing atrial fibrillation based on the statistical analysis, wherein X is at least 20 and Y is at least 50.

In an embodiment, there is a non-transitory computer readable medium having recorded thereon, a computer program for executing at least a portion of a method, the computer program including code for statistically analyzing first data based on phase gradients for at least 150 locations on a surface of a chamber of a human heart and code for identifying a plurality of locations from the at least 150 locations, based on the statistical analysis of the first data, that should be targeted for treatment.

### BRIEF DESCRIPTION OF THE DRAWINGS

FIGS. 1-5 depict exemplary comparisons between normal electrical wave propagation in a normal functioning heart and electrical wave propagation in a heart afflicted by atrial fibrillation.

FIGS. 6 and 7 present exemplary flowcharts for exemplary methods according to an exemplary embodiment.

FIGS. 8a and 8b depict an exemplary catheter used in some embodiments.

FIG. 9a is a schematic representation of a system embodiment showing a catheter in the left atrium.

FIG. 9b is a schematic representation of an atrial electrogram from one electrode.

FIG. 10 shows a schematic diagram of a catheter in a heart and additional recording, control and processing devices that are required for inverse endocardial mapping.

FIGS. 11-14 show exemplary potential maps over time for an exemplary scenario;

FIG. 15 presents pre-processing information according to an embodiment.

FIG. 16 presents potential to phase conversion information according to an embodiment.

FIGS. 17-20 show exemplary phase maps over time for an exemplary scenario.

FIGS. 21-22 show conceptual location teachings according to an embodiment.

FIG. 23 shows an exemplary phase gradient map in an exemplary scenario.

FIG. 24 shows actions of an embodiment of the method where (a) a representative potential distribution is sampled at internal points on a circle and (b) the potential distribution within the circle is reconstructed by a forward solution using potentials interpolated around the virtual inner circle from the sampled potentials.

### DETAILED DESCRIPTION

The teachings herein relate to identifying heart tissue/heart cells of interest in a living human, which tissue/cells have an association with the occurrence of atrial fibrillation. The teachings herein also relate to methods and procedures for altering the heart/implementing a surgical procedure on the heart to at least partially alleviate or otherwise reduce the occurrence and/or symptoms of atrial fibrillation.

In an embodiment, electro-anatomic mapping is used to guide exemplary treatments of heart rhythm disturbances. This can involve the following actions: i) 3D heart surface geometry is reconstructed for the chamber (or chambers) of concern; ii) electrical signals (time varying electric potentials) are recorded at a number of registered points on the heart surface; iii) electrical activity throughout the region is rendered, in time and space; and iv) statistical analysis is implemented. Based on this information, likely sources of rhythm disturbance in the heart wall are then located and, in some embodiments, ablated.

Embodiments can include the use of real time and near real time mapping and analysis of electrical activity in persistent and permanent atrial fibrillation using intracardiac catheters that record electrical activity simultaneously at multiple 3D locations.

In some embodiments, acquisition, analysis and visualization processes can be completed within 30, 25, 20, 15, 10, 5, 4, 3, or 2 seconds, or any value or range of values therebetween in 0.1 second increments (e.g., 4.4, 3.9, 3.3 to 7.8 seconds, etc.)

In some embodiments, there is the use of flexible multi-electrode basket catheters that make direct contact with the atrial surface or comes into close proximity with the heart surface(s). Electrical activity can be mapped throughout the cardiac cycle while the electrodes remain in contact with the chamber wall and/or are located in the chamber and their 3D position is known. The source of rhythm disturbances can also be identified while the electrodes are in the chamber, or within 20, 15, 10, 5, 4, 3, 2, or 1 minutes, or any value or range of values therebetween in 0.1 minute increments of the removal of the electrodes from the chamber (or movement of the electrodes to another portion of the chamber-embodiments include using standard catheters to read potentials at multiple regions within a chamber to harness the accuracy of a tightly spaced arrangement of electrodes while using conventional readily available electrode catheters (e.g., those with 64 or 128 electrodes)).

In some embodiments, the Constellation catheter (Boston Scientific, Inc.) basket catheter with 64 electrodes to record potentials is used to obtain potential readings within the chamber/on the surface of the chamber.

Conversely, some embodiments use noncontact mapping methods to obtain potentials within the heart. Here, electrical activity is measured on a surface adjacent to the inner or outer surface of the cardiac chamber of interest and is then mapped onto the heart surface in question using inverse problem techniques.

In some embodiments, St. Jude Medical, Inc. catheters and mapping system intended for noncontact 3D electro-anatomic mapping are used to obtain the potentials within the chamber. The catheter has a 64-electrode array mounted on an inflatable balloon.

In some embodiments, an Acutus Medical, Inc. mapping system based on an expandable basket catheter that contains 42 electrodes as well as ultrasound probes can be used to obtain data within a heart. With this approach, electrical activity recorded with a multi-electrode basket catheter in an atrial cavity is used to estimate an equivalent electrical dipole distribution within the atrial wall. In some embodiments, a Cardioinsight Technologies, Inc. system is used to map electrical activity measured on the body surface with a multi-electrode vest onto the epicardial surface of the heart using an inverse method. The approach is non-invasive, but it requires accurate 3D anatomic representations of body surface and epicardial geometry using computed tomography (CT) or magnetic resonance imaging (MRI).

But embodiments are not limited to the above noted catheters or even the specific features associated therewith. Embodiments include using data from a device having less than or more than or equal to 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 450, 500, 550, 600, 700, 800, 900, 1000, 1250, 1500 or more or any value or range of values therebetween in 1 increment (e.g., 23, 38, 22-66, etc.) number of channels electrodes (at least read electrodes). Any device, system or method that can enable utilitarian data collection can be used in some embodiments providing that the art enables such.

Embodiments include utilizing methods for determining physiological information for an internal body surface, such as an endocardial surface.

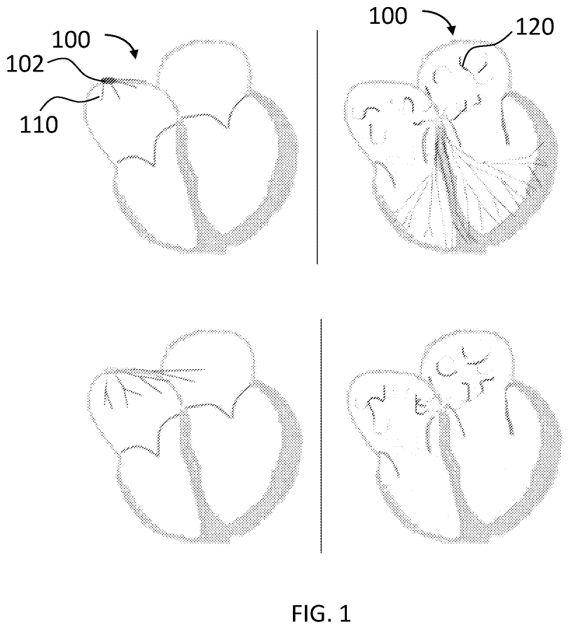

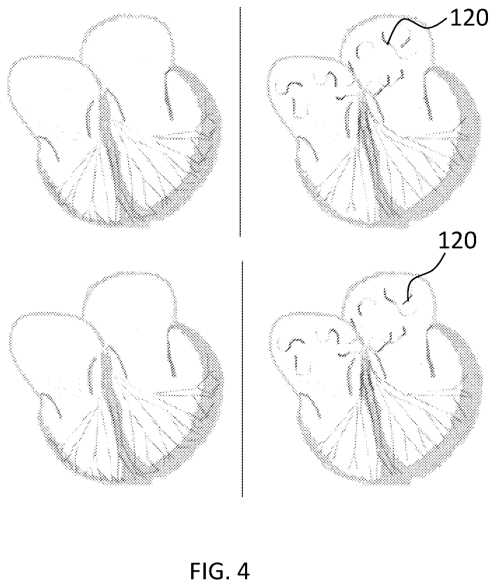

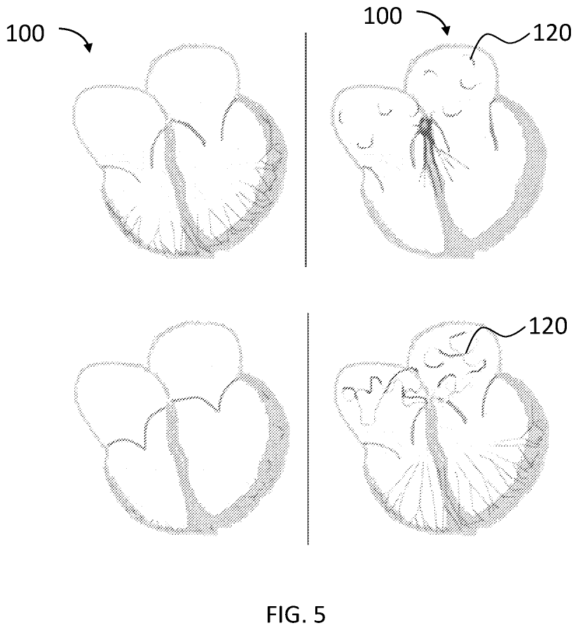

FIGS. 1-5 show general schematic representations of features of two human hearts 100. The heart on the left presents the sinus rhythm, and the heart on the right presents atrial fibrillation. The heart on the left is a normal beating heart, where regular impulses 110 produced by the sinus node 102 emanate therefrom as shown. The heart on the right is a heart beating under atrial fibrillation, where the impulses 120 are shown in a manner to represent by way of conceptual example the chaotic nature thereof. The heart on the right is meant to represent the most common heart rhythm disturbance scenario. The heart on the left shows the control heart, where the normally regular spread of electrical activation across the atria can be compared to the rapid chaotic rhythm with intermittent transmission of activation to the ventricles shown in the right (which replaces the normal regular spread on the left). This results in the irregular and often rapid heart rate that increases the risk of stroke and can limit heart function in some scenarios.

As shown from FIGS. 1 to 5, the impulses traveling through the heart in the atrial fibrillation scenario appear at first glance to be quite random. Indeed, most potential/current (electrical potential/electrical current) mapping systems and techniques result in data sets that are ambiguous at best. In some instances, some correlation can be deduced for short period of time, but often, the correlation is not replicated. The teachings herein go beyond the mere mapping of the electrical potentials within the heart, which teachings can provide a platform for identifying ambulation targets in a heart afflicted with atrial fibrillation.

The teachings herein are directed to providing an interventional treatment of persistent and permanent atrial fibrillation, or at least providing an identification of heart cells/tissue that are causing or at least implicated in the atrial fibrillation. The teachings herein can be directed to proving the interventional treatment (or the identification) to sustained episodes of atrial fibrillation that do not spontaneously terminate within two weeks. Embodiments can be directed to episodes that do not spontaneously terminate in 1 week, or 3, 4, 5, 6, 7, 8, 9, 10, 11, 12, 13, 14, or 15 days, or any value or range of values therebetween in 1 day increments.

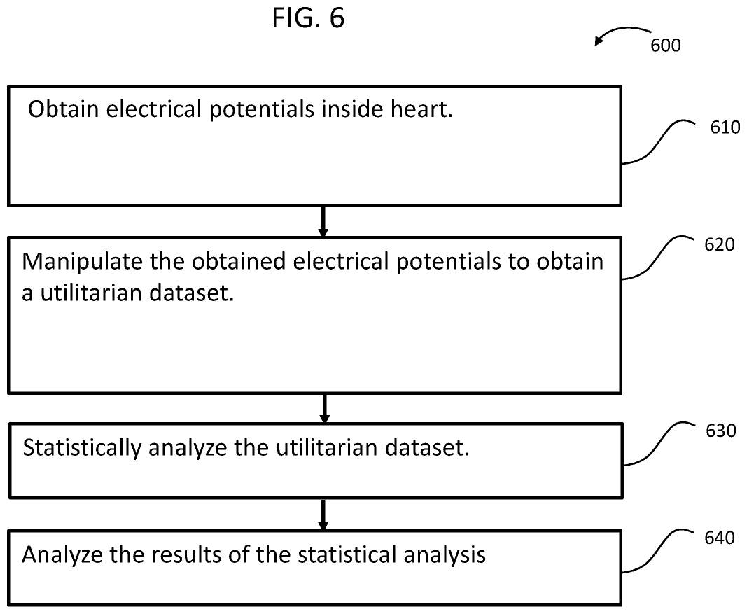

FIG. 6 presents a high level flowchart for an exemplary method, method 600, according to an exemplary embodiment. Method 600 includes method action 610, which includes the action of obtaining electrical potentials inside a human heart while the human heart is beating and thus while the human associated with the human heart is alive. Corollary to this is that in at least some exemplary embodiments, at least those directed to treating atrial fibrillation, the obtained electrical potentials in method action 610 are obtained while the heart is experiencing a sustained episode of atrial fibrillation that has not spontaneously terminated within the past few days or weeks for that matter. In this exemplary method, the heart is a heart that is in a scenario of permanent atrial fibrillation. But it is briefly noted that at least some exemplary embodiments herein can utilize the teachings detailed herein to analyze healthy tissue or otherwise analyze parts that are not experiencing atrial fibrillation.

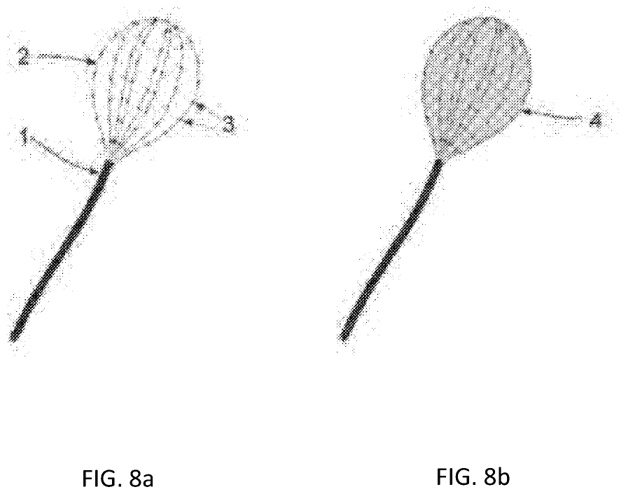

Some exemplary embodiments include obtaining electrical potentials inside the heart utilizing a basket catheter. For example, FIG. 8a shows a schematic representation of a multi-electrode mapping catheter 1. It includes multiple expandable splines 2 with sensors or electrodes 3 spaced evenly along the splines. The catheter is open in the sense that fluid such as blood for example, can pass freely between the splines. However, as shown in FIG. 1b, in this exemplary embodiment, all electrodes lie on a continuous virtual surface 4 that is closed in the mathematical sense.

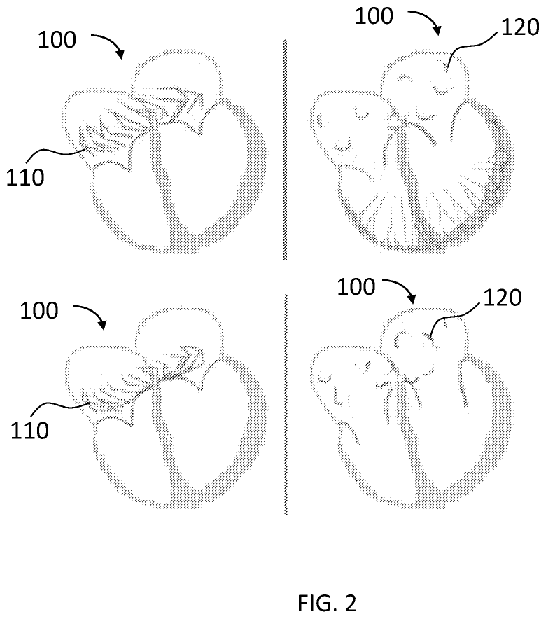

FIG. 2a shows a schematic representation of the mapping problem in a heart 5. The catheter 1 is located in the left atrium (LA), and electrical potentials generated by electrical activity in the heart can be recorded by each of the multiple electrodes 3 simultaneously. An electrogram (potential as a function of time) at a typical electrode 3 is displayed for a single cardiac cycle in FIG. 2b. The potential distribution on the LA endocardial surface 6 at successive instants through the cardiac cycle can be reconstructed based on the corresponding potentials recorded at the multiple catheter electrodes. This can be executed using an inverse approach, or solving an inverse problem. The objective of the inverse problem in some embodiments is to reconstruct source information (e.g., atrial endocardial potentials) from the measured field (e.g., potentials recorded at the catheter electrodes) based on a priori information on the physical relationships between sources and measured field. In this setting, information is also required about the 3D geometry of the endocardial surface and the 3D location of each of the electrodes. This information can be obtained using a CT scan while the catheter electrodes are in the heart chamber or some other form of imaging technique, such as using radioactive beads, etc. Any device, system, and/or method of correlating the location of the catheter to locations on a chamber of a heart that can enable the teachings detailed herein can be used in at least some embodiments. The idea is to obtain a spatial relationship, whether it be for example in cartesian coordinates or polar coordinates or radial coordinates (and thus typically in three dimensions), between the electrodes and locations on the surface of the heart chamber so that the data obtained from the electrodes can be correlated to specific and discrete locations on the surface of the heart chamber. In an embodiment, the accuracy is within plus or minus 1 cm, 0.75, 0.5, 0.4, 0.3, 0.2, 0.1, 0.08, 0.06, 0.04, 0.02, or 0.01 cm, or any value or range of values therebetween in 0.01 cm increments.

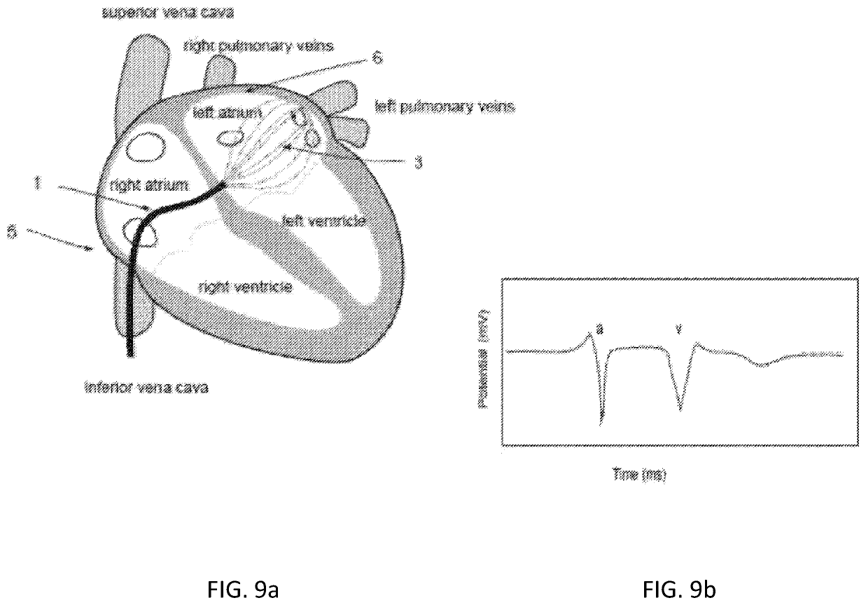

FIG. 9a shows the four cardiac chambers: the left atrium (LA), right atrium (RA), right ventricle (RV) and left ventricle (LV). An endocardial surface 6 is typically at least part of the surface of one of the chambers of the heart. Where discussed herein the endocardial surface may be represented as a 2D surface, but it is understood that a user of the system would typically be investigating a 3D endocardial surface enclosing a chamber within. In some embodiments an endocardial surface may be only a portion of a chamber, that portion being of interest.

Moreover, empirical research has shown that the further apart the electrodes are from each other, owing to, for example, expansion of the splines of the catheter, the less accurate the ultimate resulting potential map of the interior surface of the chamber. Embodiments include utilizing a catheter where the electrodes are no more than 5, 4.5, 4, 3.5, 3, 2.5, 2, 1.5, 1, 0.75, or 0.5 mm, or any value or range of values therebetween in 0.1 mm increments. Embodiments thus can include the controlled or otherwise limited expansion of the catheter splines to values that are lower than that which would otherwise be possible within a given chamber. By way of example only and not by way limitation, if the catheter was capable of expansion to the point where the electrodes would be 4 mm away from each other, the expansion might be limited to an expansion where the electrodes are only 2.5 mm away from each other, at most. This may not result in adequate data from the electrodes to map the entire surface of the chamber. However, it may not be necessary to map the entire chamber, and, alternatively, owing to the specific nature associated with implementing the teachings detailed herein, the catheter can be moved to another location within the chamber, and potentials can be obtained, and that particular region of the surface can then be mapped, and this can take place in a serial fashion for other locations within the chamber, and thus other locations on the surface. Put another way, embodiments can include obtaining the potentials within the chamber in one fell swoop for all locations on the surface of the chamber, and embodiments can include obtaining potentials within the chamber in a serial manner at different locations within the chamber for different regions of the surface of the chamber. This latter method can provide a more accurate data set, because the electrodes are closer to each other, which more accurate data set will provide more accurate potential mapping of the locations on the surface of the chamber.

By way of example only and not by way of limitation, in an exemplary embodiment, the catheter can be inserted to be proximate a first region of the chamber, and can be controlled to expand the splines of the catheter to a point where the electrodes extend from each other but within the utilitarian distances detailed above her other utilitarian distances. The electrical potentials can be recorded for utilitarian time periods, such as for example, over at least and/or no more than 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 60 seconds or more, or any value or range of values therebetween in one second increments, where consecutive electrical potentials are recorded through a recording cycle that operates at at least 500 or 1000 or 1500 or 2000 or 3000 or 4000 or more hertz or any value or range of values therebetween in 1 Hz increments. The resulting data set obtained for the temporal period where the electrodes are at the given location within the chamber can be stored and/or manipulated or otherwise used to implement at least some of the teachings detailed herein, and then the catheter can be moved to a different location within the chamber, and, if the splines were contracted, the splines can be re-expanded to obtain utilitarian spacing of the electrodes, and then the data collection can be repeated at this new location within the chamber to obtain the data set that can be utilized to develop an accurate potential map for this new region of the surface of the chamber, and this movement/data collection series of actions can be repeated however many times needed to obtain accurate data and/or accurate potential mapping of the desired regions within the heart chamber. In an exemplary embodiment, the catheter is utilized with electrodes spaced at the aforementioned limits for example to map at least 50, 55, 60, 65, 70, 75, 80, 85, 90, 91, 92, 93, 94, 95, 96, 97, 98, 99, or 100% of the surface area of the chamber or any value or range of values therebetween in 1% increments in accurate manner.

It is briefly noted that imaging of the catheter spline, or more specifically, the electrodes and the cardiac chamber surface, showing the electrodes and the surface of the chamber, and/or coordinate data indicating the relative locations between the electrodes and the surface of the chamber, will be obtained each time that the catheter is moved to a new location within the chamber to obtain data that will be used to develop a map of the potentials on the surface of the associated region of the chamber.

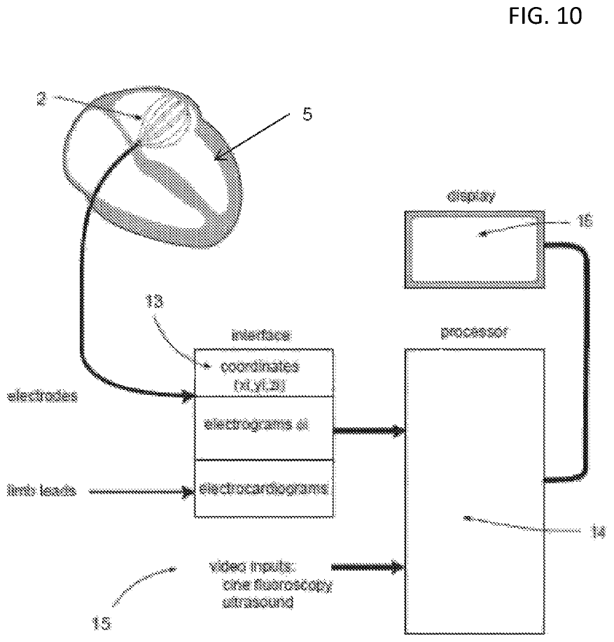

To be clear, some exemplary embodiments include any device system and/or method that can enable electric potentials within a chamber of a heart, whether on a surface of the chamber or spaced away from the chamber, to be obtained in a utilitarian manner to implement the teachings detailed herein can be utilized in at least some exemplary embodiments providing that the art enable such. But briefly, FIG. 10 shows an exemplary system that can be used to obtain the electrical potentials from within the heart chamber and/or develop at least some of the data herein. Additional details of this will be described below, but briefly, the utilization of the arrangement of FIG. 10 to execute one or more of the method actions detailed herein. Thus, in some embodiments, the arrangement of FIG. 10 is configured, such as via firmware and/or software and/or hardware, to implement one or more the method actions herein. FIG. 10 can have a control unit configured to implement one or more or all of the functionalities detailed herein and/or method actions detailed herein. Also, parts of FIG. 10 can be bifurcated and/or trifurcated and spatially located remotely provided that such enables the teachings herein.

Again with reference to FIG. 6, method 600 further includes method action 620, which includes the action of manipulating the obtained electrical potentials to obtain a utilitarian data set. In an exemplary embodiment, additional details of which will be described below, method action 620 is executed by implementing electrical potential mapping techniques further described below, but briefly, any one or more of the potential mapping teachings disclosed in U.S. Pat. No. 10,610,112, issued on Apr. 7, 2022, naming Bruce Smaill as an inventor, and naming Auckland UniServices Limited of New Zealand as the Applicant, can be used in some embodiments to obtain values for electrical potentials for locations on a surface of a heart chamber. By way of example, the inverse mapping techniques disclosed in the aforementioned patent can be utilized in some embodiments to develop a potential map of the surface or portions of a surface of the chamber of interest. But to be clear, and device system and/or method that will enable electrical potential mapping of potentials obtained from electrodes onto the surface of the chamber can be utilized in at least some exemplary embodiments, providing that the art enable such.

Method action 620 further includes, at least in some embodiments, preprocessing of the data obtained from the potential mapping of the surface. Under the rubric of manipulating the obtained electrical potential to obtain a utilitarian data set, embodiments further include implementing cardiac tissue cell phase mapping techniques utilizing the data obtained from the execution of the potential mapping technique detailed above. Some additional features of this will be described below, but briefly, by way of example, a Hilbert transform can be utilized to develop instantaneous phase data from the potential data such as that disclosed by Pawel Kuklik et al in Reconstruction of Instantaneous Phase of Unipolar Atrial Contact Electrogram Using a Concept of Sinusoidal Decomposition and Hilbert Transform, published in IEEE Transactions On Biomedical Engineering, Vol. 62, No. 1, January 2015. But to be clear, any other transformation technique or any other device system and/or method that can enable these data to be developed from the electrode potential readings and/or from the surface potential data can be utilized in at least some exemplary embodiments providing that the art enable such.

Method 600 further includes method action 630, which includes the action of statistically analyzing the utilitarian data set obtained in method action 620. In an embodiment where the utilitarian data set is a phase map or otherwise constitutes phase data of specific locations on the surface of the chamber, over a utilitarian time period, such as, for example, 10 or 15 or 20 seconds as noted above, which utilitarian time period can correspond to the timing of the readings of the electrical potentials utilizing the electrodes located in the chamber, the action of statistically analyzing the utilitarian data set can include time averaging maximum phase gradients between the different locations on the surface of the chamber.

Method 600 further includes method action 640, which includes the action of analyzing the results of the statistical analysis executed in method action 630. In an exemplary embodiment, as will be described in greater detail below, chamber surface locations that have a statistically meaningful phase gradient can be considered locations where there exists heart tissue that is playing a role in causing atrial fibrillation of the heart, at least relative to other tissue of the heart. In at least some exemplary embodiments, at least some of these locations having the nonzero phase gradient can be considered for targeting in an ablation process.

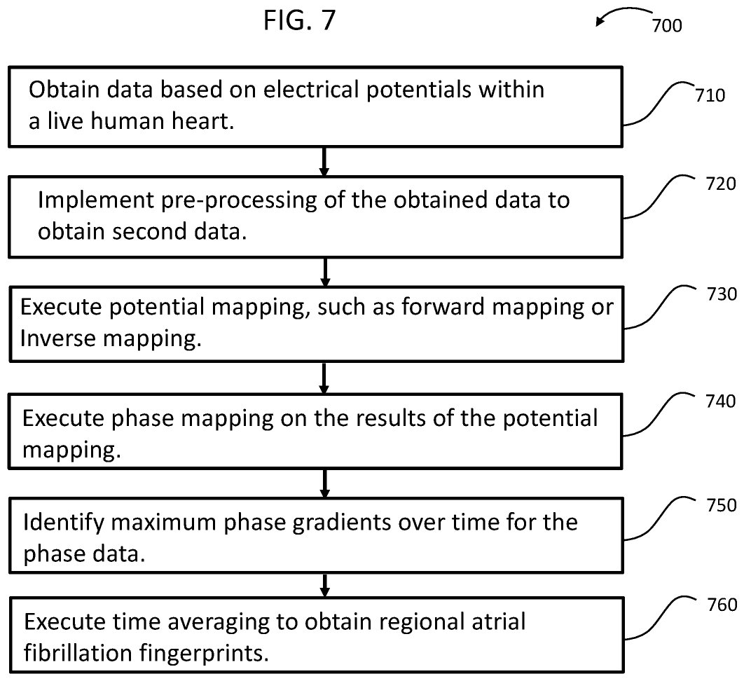

FIG. 7 provides a flow variation of an exemplary method, method 700, according to an exemplary embodiment. This method does not specifically require the actor to obtain the electrical potentials from within the heart chamber. Instead, another actor could obtain the information and otherwise provide the information to another actor executing method 700. In this regard, method 700 could be executed remotely from the patient otherwise from the operating room where the electrical potentials are being recorded. By way of example only and not by way limitation, in an exemplary embodiment, an Internet connection or a telephone connection or some other form of relative high-speed data communication system can be utilized to transfer the role signal potentials and or the spatial location data associated with the electrodes relative to the surface from the operating room or whatever hospital or location where the human patient is being treated or otherwise where the human patient is located during the action of obtaining the electrical potential within the heart, to a remote location, such as where a server or a remote computer is located, which could be tens or hundreds or thousands of kilometers away, in this remote computer remote server could implement method 700. Accordingly, embodiments include methods of practicing remote treatments or remote analysis and/or devices and/or systems that enable such, such as by way of example only and not by way limitation, a laptop and or a desktop computer or some other type of computer system, such as a smart phone for that matter, located otherwise co-located with the patient, that can receive the data from the electrodes or otherwise receive the data based on the data from the electrodes, and transform this data into a communicator ball medium which can be communicated over the Internet or over a phone line etc. to the remote location, where method 700 could be executed. Corollary to this is that at least some exemplary embodiments include some form of computing system, such as one or more of the aforementioned systems, that can receive the transferred data and execute method action 700. Moreover, some embodiments include the ability to then send the results of method 700 back to the location where the patient is located so that an ablation treatment procedure for example can be implemented based on the result of method action 700 and/or any one or more the additional actions detailed herein. But of course, an embodiment includes a system that is located with the patient that can execute method 700.

In at least some exemplary embodiments, all of the proceeding paragraphs, minus the actual treatment, can take place in at least some exemplary embodiments, within 30, 25, 20, 15, 14, 13, 12, 11, 10, 9, 8, 7, 6, 5, 4, 3, or 2 minutes, or any value or range of values therebetween in one minute increments. And in some embodiments, the treatment will add no more than 10, 15, 20, 25, 30, 35, 40, 45, 50, 55, 60, 70, 80, or 90 minutes to those times.

And returning to FIG. 7 and method 700, method 700 includes method action 710, which includes the action of obtaining data based on electrical potentials within a live human heart. As just noted, method action 710 does not require per se the actual action of utilizing the electrodes located in the heart. Method action 710 can instead be executed by obtaining a data set or otherwise obtaining data based on those readings from the electrodes. For example, method action 710 can be executed by receiving over the internet a data package or a series of data sets or a single data set indicating the time based electrical potential values on one or more or all of the electrodes of the catheter and or the accompanying spatial relationships between the electrodes and the surface of the heart chamber.

The dataset could be a set of raw electrical values, or could be data extrapolated from the raw electrical values (e.g., a normalized set of electrical potentials, or pre-processed electrical potentials, or electrical potentials where extraneous values are omitted or smoothed, etc.). As used herein, “data based on X” means X or data that is extrapolated from X or data that is extrapolated from data extrapolated from X.

But still, with respect to embodiments where the system, such as a computer system, is implementing method 700, method action 710 could be executed by receiving the electrical values directly from the electrodes via leads extending from the catheter to the computer system utilized to execute method action 700.

In an embodiment, the obtained data can be time based data (such as electrical potential readings) for ABC number of electrodes, where for respective electrodes, there are discrete values in time increments of at least 200, 500, 750, 1000, 1250, 1500, 2000, 2500, 3000, 3500, 4000, 4500, 5000 Hz (the numbers need not be the same for each electrode), over at least, or equal to or no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, or 90 seconds (including consecutive seconds) or any value or range of values therebetween in 1 second increments. Thus, the data could be, for a 64 electrode catheter (by way of example only and not by way of limitation, a 64 channel Constellation™ basket catheter available as of Jan. 10, 2022, at the Royal Melbourne Hospital) and a system taking measurements at 2,000 Hz over a 13 second period, 1,664,000 time based values. And note that that might be for one location. But method action 710 could also be executed in one fell swoop, to cover multiple readings from multiple locations, and thus there could be potentially two or three or four or five or more times that number of values. Still, in at least some exemplary embodiments, it is envisioned that electrode readings for one location within the chamber will be obtained and transferred to the computer for the execution of method 700, or at least one or more method actions therein, such as the execution of method 720 and/or method 730, and then the catheter will be moved to obtain additional electrical potentials, which will then be sent to the computer, and so on.

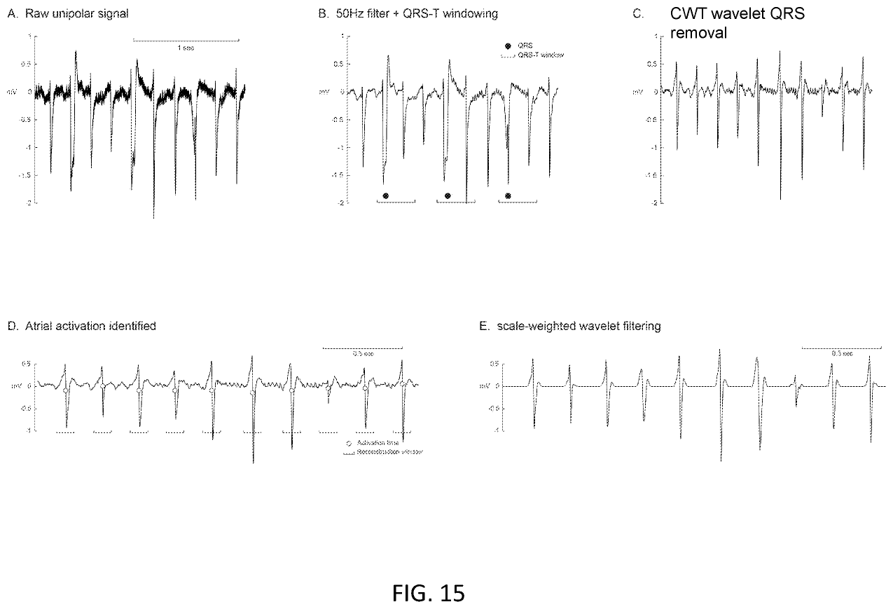

Method 700 further includes method action 720. This can include implementing a preprocessing of the obtained data to obtain second data. It is briefly noted that method action 720 is optional in some embodiments and/or is otherwise a method action that can be practiced in various extremes or lack thereof. FIG. 15 shows some conceptual data associated with addressing scenarios where the signals from the electrodes is noisy and contaminated by the asynchronous electrical activity of the ventricles. One or both of these aspects can be subtracted, using a suite of robust wavelet-based filtering applications that enable the recovery of the underlying atrial electrograms.

And note that this can be an example of where the data of method action 710 can be data based on electrical potentials within a live human heart. In this regard, in an exemplary embodiment, method action 720 could be executed by the hospital or another actor in a scenario where, for example, method 700 is executed by some remote facility located remotely from the patient (in which case for example method action 720 would not be part of method 700, and thus an abbreviated version of method 700 would be practiced). In any event, this is but one example of how data based on electrical potentials within a live even heart could come about, which data based on electrical potentials could be the data obtained in method action 710.

Method 700 further includes method action 730, which can include executing potential mapping, such as forward mapping or inverse mapping, of the surface of the chamber in which the electrodes are or were located. By way example only and not by way of limitation, any one or more the techniques detailed in the aforementioned US patent noted above, U.S. Pat. No. 10,610,112, can be utilized in at least some exemplary embodiments. Briefly, we will focus on inverse mapping techniques. That is, some embodiments use inverse potential mapping. This can be done even though at least some of the electrodes, including more than 20, 30, 40, 50, 60, 70, or 80% or more of the electrodes used to obtain the potential values are not in contact with the atrial wall. Inverse mapping can account for this and be used to reconstruct a time-varying potential field across the 3D surface, such as the left atrial chamber surface.

Here, the second data obtained using the pre-processing action of method 720 is used in the potential mapping of method action 730.

Embodiments include systems to enable reconstruction of panoramic electrical activity in a heart chamber from physiological information, most particularly, time-varying electrical potentials (which may also be referred to as electrical fields or simply, fields) recorded using an open catheter inside the chamber that contains multiple sensors which may comprise electrodes, some or all which are not in contact with the wall of the chamber. In some embodiments, a numerical approach can be used to estimate physiological information (such as electrical potentials, electrical fields, or fields) in the volume bounded by the electrodes from the recorded potentials. This provides the additional boundary conditions necessary for accurate inverse mapping of potentials onto the inner surface of the heart chamber. For instance, in inverse solution packages that employ Boundary Element Methods (BEMs), it is utilitarian to specify both potential and potential gradients at measurement points.

But some embodiments utilize meshless methods to address the inverse problem represented in FIG. 2. There, a set of fictitious current sources (or sinks) is positioned on a boundary outside the cardiac surface and it is assumed that Laplace's law holds throughout the volume inside the boundary. Source magnitudes which give rise to potentials that best match potentials recorded on the catheter are then determined using standard inverse solution methods. Corresponding potentials on the cardiac surface are then estimated from these sources. Meshless methods can be efficient and robust in this setting, and can be much simpler computationally, are far less reliant on accurate 3D descriptions of the endocardial geometry of the heart cavity and provide solutions when measurement points lie on the endocardial surface relative to the numerical approach described above.

This system can enable rapid reconstruction and visualization of electrical potentials on an internal body surface, particularly an internal surface bounded by a chamber such as the endocardial surface of a cardiac chamber, or region of that chamber. These potentials comprise physiological information and can be, in some embodiments as noted above, from electrical potentials which may be measured with an expandable multi-electrode basket catheter, in which either all or some of the electrodes are not in contact with the surface. Such a catheter is open in a sense that bodily fluid such as blood within the chamber passes freely through it, but in which the electrodes define a mathematically closed 3D surface.

It is anticipated that the system or methods disclosed could be used as the basis of a complete mapping system or alternatively to enhance the performance of existing or proposed systems.

Some exemplary methods obtain exemplary potential maps, will now be briefly described.

An exemplary method of determining physiological information for an internal body surface using an open catheter comprising multiple electrodes bounding a volume within the catheter can include:
a) obtaining a first set of electric potentials using a plurality of the electrodes,
b) determining a first set of boundary conditions from the first set of electric potentials,
c) using the first set of boundary conditions to perform a forward solution to a first set of differential equations to provide a second set of electric potentials within the volume,
d) determining a second set of boundary conditions from the second set of electric potentials,
e) using the second set of boundary conditions to perform an inverse solution to a second set of differential equations, and
f) determining the physiological information for the internal body surface using the inverse solution.

The method can also include interpolating the first set of electric potentials. The physiological can comprise electric potentials on the internal body surface. The internal body surface can comprise an endocardial surface. The endocardial surface can comprise, least in part, an atrium or ventricle. The physiological information can comprise an electro-anatomical mapping. The method can comprise using a numerical method to solve the first or second set of differential equations. The numerical method can comprise any one or more of a finite element method, a boundary element method, or a meshless method. The numerical method can be implemented using a processor. The open catheter can comprise a flexible basket. The method can comprise positioning the catheter within a chamber bounded by the internal body surface. The method can comprise positioning the catheter proximal to a region of the internal body surface. The method can comprise positioning the catheter in a plurality of positions within the chamber. The method can comprise locating the catheter in a first position to obtain a first set of physiological information for a first portion of the internal body surface, and at least one second position to obtain a second set of physiological information for a second portion of the internal body surface.

The method can comprise introducing the catheter into the body using a percutaneous technique.

The method can be a method of determining physiological information for an internal body surface of a human using an open catheter comprising multiple electrodes bounding a volume within the catheter, the method comprising:
a) obtaining a set of electrode electric potentials using a plurality of the electrodes;
b) determining a boundary that contains the internal body surface;
c) determining a set of discrete fictitious sources on the boundary;
d) using inverse solution methods, determining source magnitudes of discrete fictitious sources of the determined set of discrete fictitious sources that give rise to potentials that sufficiently match the obtained electric potentials;
e) determining corresponding potentials on the internal body surface of the human from the determined source magnitudes; and
f) using the determined corresponding potentials on the internal body surface, determining the physiological information for the internal body surface.

In some embodiments, the inverse solution methods are standard inverse solution methods.

In some embodiments, the internal body surface is an endocardial surface of a cardiac chamber.

The method can comprise introducing the catheter into the human body using a percutaneous technique.

The action of determining the boundary surface is executed before, during and/or after the action of obtaining the set of electrode electric potentials.

In some methods, the inverse solution methods are standard inverse solution methods.

In some methods, the methods include obtaining the geometry of the internal body surface, wherein the internal body surface is a heart chamber and obtaining data indicating the position of the catheter within the heart chamber.

The method can include creating a visual representation of the heart chamber based on data indicative of the electrode electrical potentials, the source magnitudes of the discrete fictitious sources, the corresponding potentials on the internal body and the geometry of the heart chamber and position of the catheter in the heart chamber. The method further can include executing spatio-temporal processing of the determined corresponding potentials on the internal body surface.

The method can include reconstructing position of the catheter relative to the internal body surface and accounting for potential error in the determined corresponding potentials on the internal body surface. The method can include displaying the determined physiological information on an image of the internal body surface. The method can include locating the catheter at a first position to ultimately obtain a global image of the internal body surface and locating the catheter at at least one second position to obtain a more accurate estimate of potentials on a portion of the internal body surface, the more accurate estimate of potentials corresponding to the determined corresponding potentials on the internal body surface. The action of determining corresponding potentials on the internal body surface can be executed by a numerical method. The action of determining corresponding potentials on the internal body surface can be executed by a meshless method.

And there can be non-transitory storage medium having machine-readable instructions stored thereon, that when executed by a processor cause the processor to perform the following actions:
determine a boundary that contains an internal body surface of a human;
determine source magnitudes of discrete fictitious sources of a set of discrete fictitious sources that give rise to potentials that sufficiently match a set of electrode electric potentials obtained from a plurality of electrodes that were and/or are located in the internal body surface; and
determine corresponding potentials on the internal body surface from the determined source magnitudes.

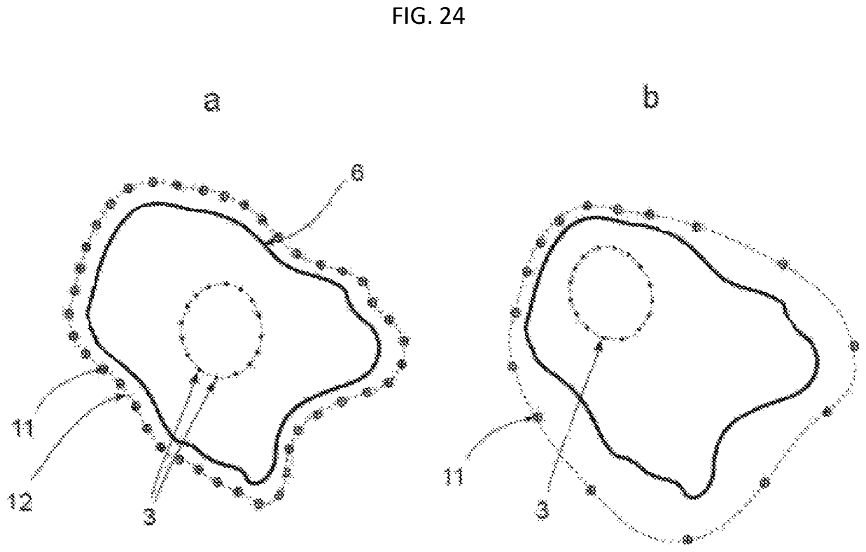

FIG. 24a illustrates how the inverse endocardial mapping problem is approached with meshless methods that use the MFS. In FIG. 24a a set of discrete fictitious sources 11 is positioned on a boundary 12 that contains the endocardial surface 6 of the cardiac chamber. The fictitious sources are each fundamental solutions of Laplace's equation (equation 1 above) within the source-free volume Ω contained in the cardiac chamber. The potential ϕᵢ at any point (x,y,z) in 3 due to a source i located on the boundary 2 is

$${ϕ}_{i} ( x , y , z ) = {α}_{i} + \frac{{β}_{i}}{{r}_{i}} \qquad ( Equation 4 )$$

where rᵢ is the Euclidean distance between (x,y,z) and the source at (xᵢ, yᵢ, zᵢ).

That is rᵢ=√((x−xᵢ)²+(y−yᵢ)²+(z−zᵢ)²)

The total potential Φ at any point in Ω contributed by a set of M fictitious sources is

$$Φ ( x , y , z ) = {\sum }_{i = 1}^{M} {ϕ}_{i} ( x , y , z ) \qquad ( Equation 5 )$$

Source densities βᵢ are selected to match potentials recorded at each of the electrodes on the catheter 4 in a utilitarian manner, but also potentials inside the catheter 5 estimated by solving the forward problem if desired. Source densities are determined by solving inverse problem with appropriate regularization and endocardial surface potentials are then mapped using equation 5.

While optimal placement of fictitious sources is empirical to some extent, a number of relatively straightforward a priori rules apply. The number of independent fictitious sources is less than the degrees of freedom of the measured potential distributions as determined by singular value analysis. The distribution of sources on the fictitious boundary should be bound by this constraint and it should be adaptive-most dense where the measurements are closest to the cardiac surface and least dense where they are furthest from it. Where sources are sparse, the accuracy of inverse mapping is increased with displacement of the surface from the cardiac boundary and vice versa. Finally the displacement of the boundary should be sufficient to accommodate uncertainty in the representation of cardiac surface geometry.

FIG. 24b represents a method of distributing fictitious sources that is consistent with these rules. The number of independent sources 11 is equal to the number of electrodes 3 on the catheter and their spacing reflects the position of the catheter with respect to the wall. It is possible to add additional sources without loss of generality by interpolating along the fictitious boundary between independent source points.

The use of meshless methods in the setting above gives rise to computationally efficient inverse solutions that are marginally less accurate than BEM, but more robust in the presence of uncertainty about endocardial geometry and the relative position of measurement sites on the catheter with respect to the endocardial surface. Finally, the positioning of independent sources on the external boundaries provides direct feedback on the spatial resolution of potentials that are mapped onto the endocardial surface.

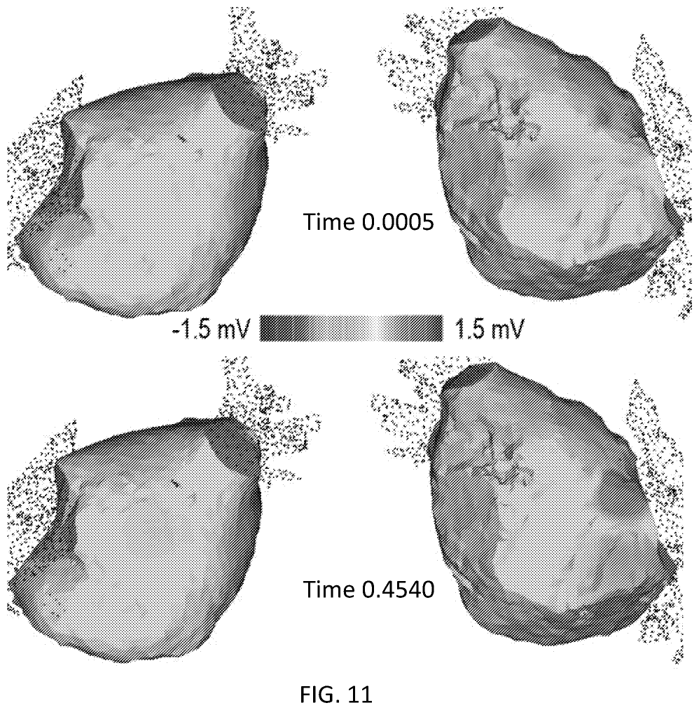

By way of example only and not by way limitation, data techniques can be used to reconstruct electrical potentials (voltages) on the inner surface of the atrial cavity from signals recorded at the individual electrodes on a basket catheter where some or all electrodes are not touching the atrial surface. The 3D geometry of the inner surface of the heart chamber (such as the left atria, with reference to FIG. 11) which can be specified and the positions of each of the electrodes with respect to this surface are also known. This information is information that can be obtained by using, for example, electrical mapping systems used to guide ablation in clinical electrophysiology laboratories, such as those at the Royal Melbourne Hospital on Jan. 10, 2022.

In an exemplary scenario, where a 64 channel catheter (Constellation™, Boston Scientific) was introduced into the left atrium (LA) by transeptal puncture of a person afflicted with permanent atrial fibrillation, method 730 can include the action of using software to execute inverse mapping based on the electrode readings. Method 730 can be executed in real-time while the basket catheter is within the atrial cavity, at least in some embodiments.

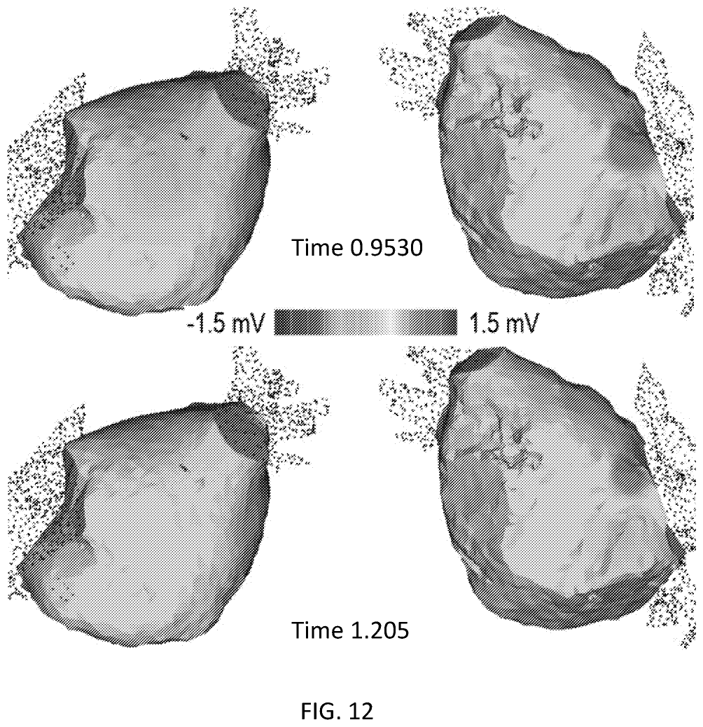

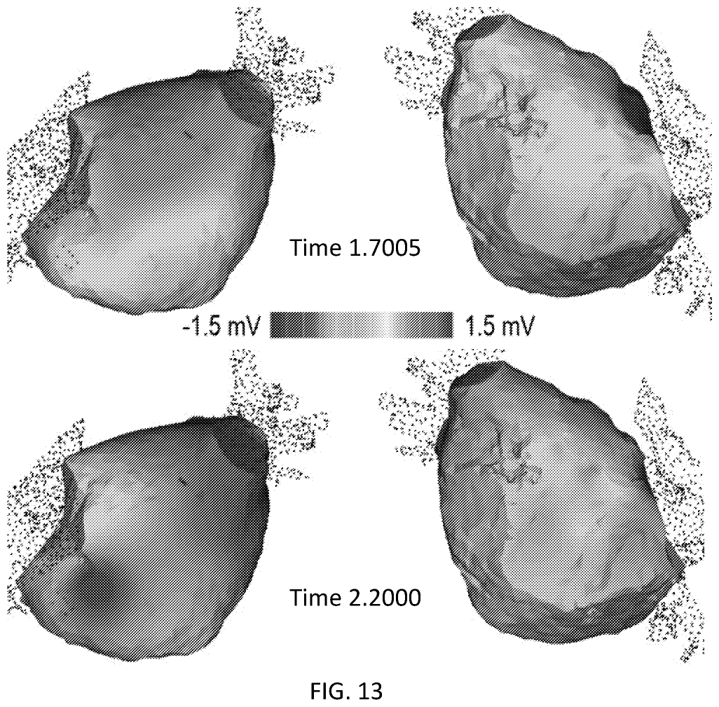

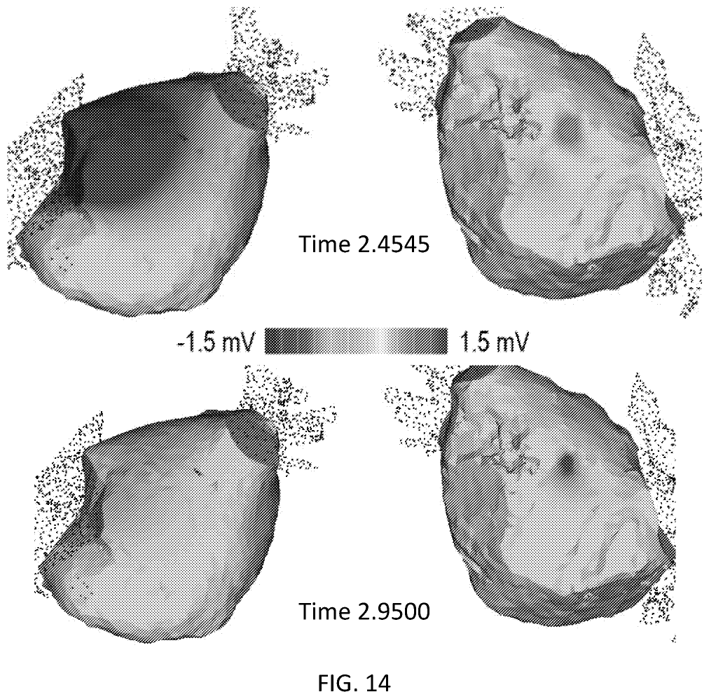

FIGS. 11-14 show by image presentation data from a result of the merging of data obtained using an inverse mapping technique (e.g., via a meshless method such as that detailed above) with data from CT images for an exemplary left atrium (posterior on the left, anterior on the right), where the figures show potential values at instantaneous points in time at locations on the surface of the left atria, and collectively show changes with some exemplary time progressions at the various locations by way of example only. More specifically, the electrode potential values corresponding to the time periods (where the monitoring device operated at 2,000 Hz-collecting readings two-thousand times per second) for the respective electrodes was, using inverse mapping, used to obtain electrical potentials on the surface of the left atria (visually represented in FIGS. 11-14). By way of example, the data from 64 electrodes can be used to develop potential values for 64, 100, 150, 200, 250, 300, 350, 400, 450, 500, 600, 700, 800, 900, 1000, 1250, 1500, 1750, 2000, 2500, 3000, 3500, 4000, 4500, 5000, 6000, 7000, 8000 or more, or any value or range of values therebetween in 1 increment locations on the surface (or surface region-again, embodiments can include utilizing the catheter with a “reduced” total volume, where the electrodes are closer together relative to that which would otherwise be the case, to obtain electrical potentials at a sublocation or a subregion within the cavity, to obtain more accurate data, and then the catheter can be moved to another subregion in the process continued), and these developed potentials being present for respective time increments at the cycle of the monitoring system (here, 2000 Hz). FIGS. 11-14 show the time varying potentials on the surface, or more accurately, at the various surface locations.

But to be clear, embodiments need not provide this exemplary any exemplary imaging. It can be sufficient to simply obtain time varying data sets for the potentials at the locations on the surface of the cavity.

It is briefly noted that there can be a point of diminishing returns with respect to the number of locations used in the inverse mapping technique. In this regard, the potentials on the surface that are calculated are limited in accuracy vis-à-vis the number of electrodes utilized in a given catheter. Thus, utilitarian value can potentially be obtained by utilizing less than the full number of locations in the inverse mapping the net which a given system could potentially develop otherwise implement. This can have utilitarian value with respect to improving computational times. That said, in some embodiments, there could be potentially more locations than those detailed above. Any number of locations resulting from the inverse mapping (or forward mapping for that matter in embodiments that utilize such) that can enable the teachings detailed herein can be utilized in at least some exemplary embodiments. Corollary to this is that any cycle time and/or total length of time that can enable the teachings detailed herein can be utilized in at least some exemplary embodiments providing that the art enable such.

The present inventors have found that mere potential mapping, while utilitarian in some instances, can be better utilized (or more specifically the data of the mapping can be better utilized), at least in some embodiments, by for example developing heart tissue cell phase data therefrom. In this regard, the potential maps are not necessarily useful in the context of the exacting nature that is required to identify heart tissue that is a driver or a substrate of atrial fibrillation, or otherwise identify tissue that can be ablated to alleviate at least some of the effects of atrial fibrillation. Indeed, the sampling of images shown in FIGS. 11-14 show clearly that the potential mapping result exhibits the chaotic activity that is typical in atrial fibrillation. The point is that it is very difficult to extrapolate what is occurring using even exacting time increment potential mapping, and on a per patient basis, typically less utilitarian than the teachings herein vis-à-vis tissue identification.

Thus, embodiments include extracting useful information, or more useful information from the electrical signals or electrograms recoded at the electrodes and/or from the potential map developed for the surface from those electrical signals.

And this leads to method action 740, which includes executing phase mapping on the results of the potential mapping. Again, as noted above, at least some of the techniques detailed in the Journal article published in IEEE Transactions On Biomedical Engineering, Vol. 62, No. 1, January 2015, by Pawel Kuklik et al, entitled Reconstruction of Instantaneous Phase of Unipolar Atrial Contact Electrogram Using a Concept of Sinusoidal Recomposition and Hilbert Transform, are used to develop the phase data. But other method actions can be used. Any method that can enable phase data to be developed that is utilitarian can be used in some embodiments.

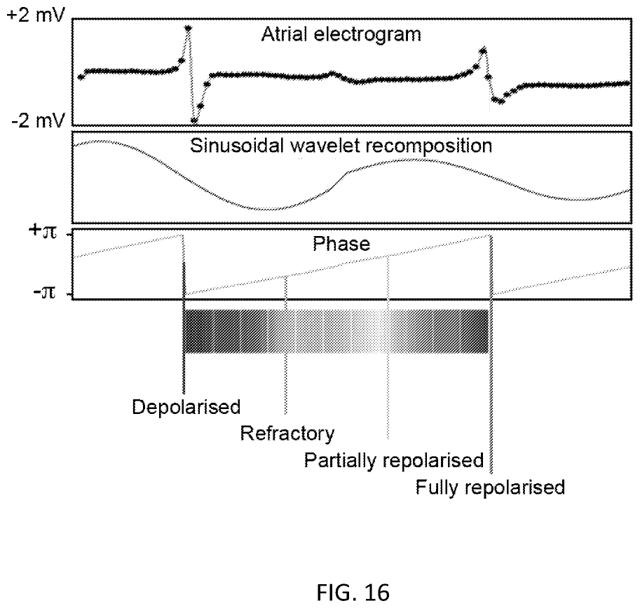

Briefly, a Hilbert transform, which can be used to transform data from the potential realm to the phase realm, is a linear operator transforming a function u(t) into a function H(u)(t)

$$H ( u ) ( t ) = \frac{1}{π} P {\int }_{- ∞}^{∞} \frac{u ( t )}{t - τ} d τ \qquad ( 1 )$$

where P is the Cauchy principal value of the integral. Phase is defined as an angle between the original signal and the Hilbert transform of the signal. Exact formulations of the phase vary between studies. Here, for example only, we define instantaneous phase as follows:

$$ϕ ( t ) = arctan ( \frac{- ( u ( t ) - u \text{*)}}{H ( u ) ( t ) - u *} ) \qquad ( 2 )$$

where u* sets the origin of the phase plane with respect to which phase is computed. Instantaneous phase increases monotonically within consecutive cycles of oscillation, reverting to a base value after completion of each cycle (by way of example). This property results in a “sawtooth” appearance of the instantaneous phase plot (see FIG. 16, more on this below).

In order to assess effects of specific features of the unipolar deflection morphology, idealized signals can be constructed using analytic considerations. This approach can enable control the morphology of the electrograms (such as the amplitude of R and S waves and the level of noise) and assessment of their effect on the reconstructed phase. Signal processing can be conducted in MATLAB (version 7.12, Mathworks Inc., Natick, MA, USA). The Hilbert transform can be calculated using the “Hilbert” function. Phase can be calculated using (2) using four-quadrant inverse tangent function “atan 2.” Methods can include a transformation of atrial unipolar electrograms that can be applied prior to application of the Hilbert transform. The transformation can be based on the following assumptions (by way of example):
1) Due to the mathematical properties of the Hilbert transform, phase reconstruction of the instantaneous phase performs best in case of a sinusoidal morphology of the signal.
2) Local activity in unipolar signals related with a beginning of new cycle is proportional to the signal slope.

Based on these considerations, the following transformation can be used:
1) The transformed signal is a sum of sinusoidal waves of one period length (called “sinusoidal wavelets” below).
2) For each time point of the original signal, one sinusoidal wavelet is created.
3) The amplitude of the sinusoidal wavelet is proportional to the slope of the signal at a given time point.
4) A wavelet is generated only if a derivative of the signal is negative (since a negative slope in unipolar electrogram corresponds with the passing of a wave).
5) The period of the sinusoidal wavelet is equal to the mean cycle length of the electrogram derived from dominant frequency of given electrogram.

These steps can be summarized in a following equation:

$$w ( t ) = {\int }_{t - \frac{T}{2}}^{t + \frac{T}{2}} {dt}^{′} sin ( t - {t}^{′} ) \vert \frac{dv}{{dt}^{′}} \vert \frac{1 - sign ( \frac{dv}{{dt}^{′}} )}{2} \qquad ( 3 )$$

where w(t) is a transformed signal, v(t) is an original electrogram, and T is a mean cycle length of the original electrogram (derived from dominant frequency of the electrogram) and sign( ) is the signum function.

Because a sinusoidal wavelets can be used to construct transformed signals, we term this transformation a “sinusoidal recomposition.” The phase of the recomposed signal can be calculated using (2). Since the recomposed signal is a sum of sinusoidal wavelets with a mean value equal to zero and wavelets of the greatest amplitude are clustered around the negative slope of the local deflection, the resultant recomposed signal also has a sinusoidal morphology oscillating around zero value. Based on this consideration, we set u* (origin of the phase space with respect to which phase is computed; see (2)) to zero. One can use a specific definition of phase presented in (2) in order to obtain timing of the phase inversion (time point at which phase changes value from maximum to minimum denoting a beginning of a new cycle) coinciding with the timing of the local deflection in the electrogram. Formulation in (2) results, in case of a sinusoidal signal, in phase inversion occurring at position of the maximum negative slope of sinusoid. Since during sinusoidal recomposition individual sinusoidal wavelets are triggered according to the timing of the negative slope in electrogram, this will result in timings of the phase inversions centered at timings of the local deflections in electrogram. (All of this by way of example to enable some exemplary embodiments.)

With respect to embodiments that utilize phase, mapping can be used to analyze atrial fibrillation, in some embodiments, the electrograms at the various locations (points) on the surface of the chamber (developed in method action 730 for example) are transformed into a phase record that represents the “time-history” of recent activation at respective locations (points). In embodiments, the transformation removes the magnitude variation that complicates interpretation of potential maps.

FIG. 16 shows a conceptual temporally consistent change of electrical potential for a given location converted into change of phase for that given location over time in a heart cell of a heart not afflicted by atrial fibrillation, but it is noted that this can also correspond to such for a heart cell in a heart afflicted by atrial fibrillation. While a Hilbert transform was used to obtain this data, other methods/regimes of transformation can be used providing that the result provides utilitarian data, as noted above. Corollary to this is that software packages can be utilized to transform the electrical potential data obtained in method action 730 to the phase data obtained in method action 740, and this software can be part of the systems detailed herein.

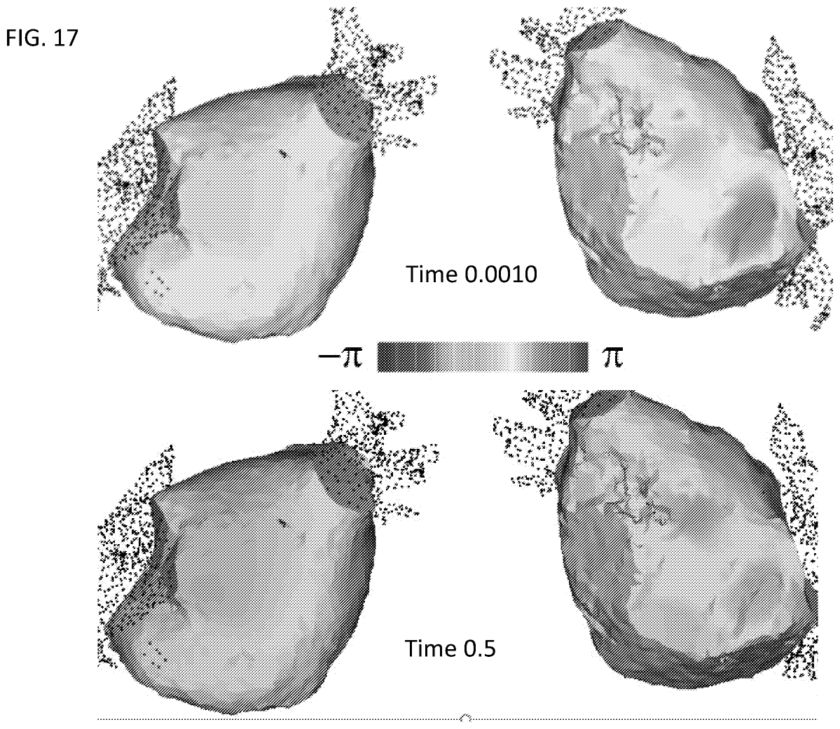

FIG. 17 shows an example of the phase of the various locations on the chamber surface at an instant in time (time 0.0010) represented in visual format. Blue indicates activation (depolarization), light blue/green shows that tissue is refractory (unable to be activated), yellow/gold (partially repolarized) indicates that activation may be able to occur although propagation is expected to be slow. Red shows fully repolarised regions that will support the normal spread of electrical activation. This information is rendered as a color map across the left atria surface that “predicts” the spread of activation. A moving wave of activation (blue) cannot enter refractory regions (green) and spreads via regions that are repolarized (red).

From the phase maps, in at least some exemplary embodiments, more consistent patterns of electrical activity that appear to repeat over time can be recognized. For instance, activation that appears to be occurring in some regions more than others can be identified, or at least more easily identified, and in some embodiments, and this can be associated with patterns of phase difference.

In any event, as a result of method action 740, in at least some embodiments, phase data for the various temporal locations associated with the potential data obtained in method action 730 for the respective locations can be developed. In an embodiment, where for example, there are 1,000 surface locations (in the potential map, and thus if one to one locations are used, the phase map), and the sample cycle was 2,000 Hz over a 13 second period, 26,000,000 time based values for phase can be obtained (each of the 1000 locations having 26,000 values in 0.0005 ms increments). But again, at least some exemplary embodiments include normalizing or otherwise removing extraneous data points, so the actual number of values will change or could change. But in any event, the ideas that while the visual images can be useful or otherwise appealing, the data obtained in method action 740 need not necessarily be in the form of a visual representation. It could be in a temporal based matrix or data set.

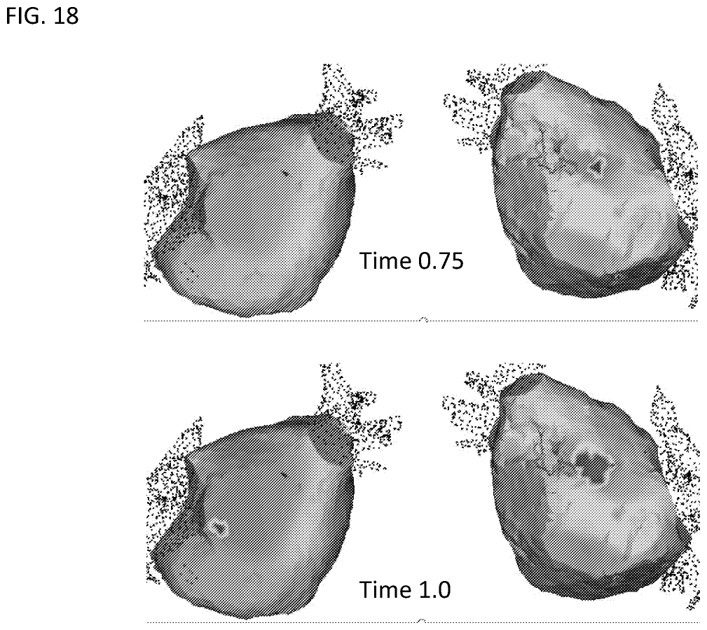

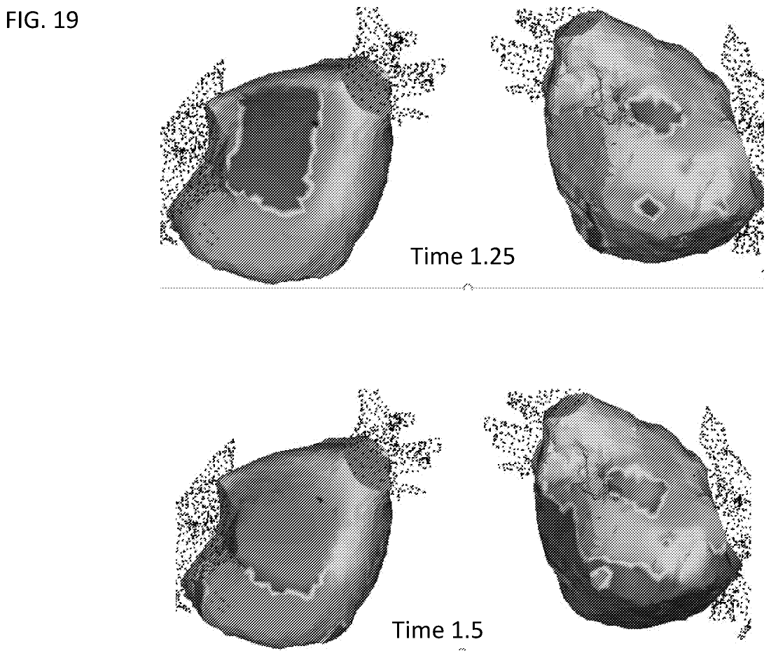

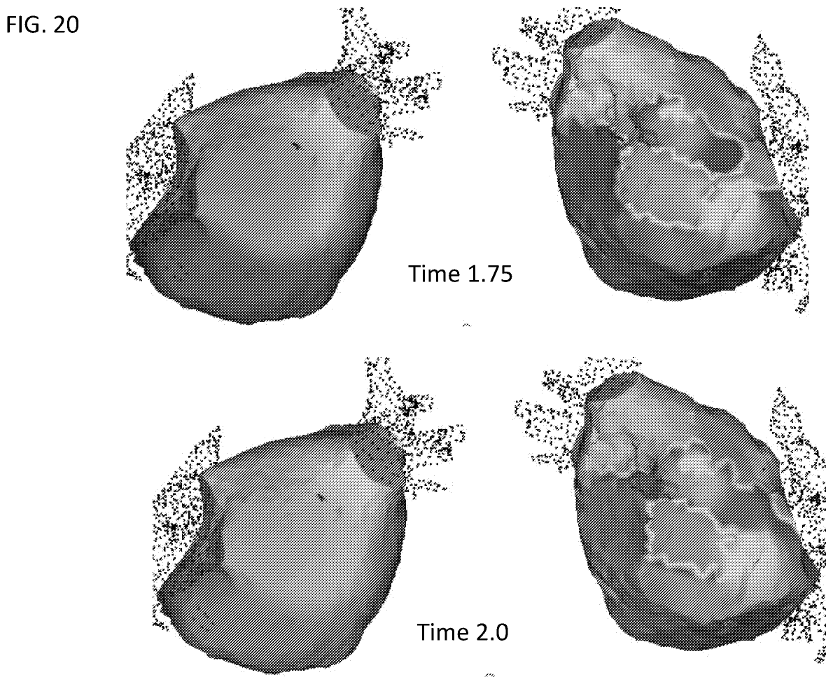

But here, FIGS. 17-20 show by image presentation data from a result of the merging of data obtained using the phase mapping technique with data from CT images for a exemplary left atrium (posterior on the left, anterior on the right), where the figures show phase values at instantons points in time at locations on the surface of the left atria, and collectively show changes with some exemplary time progressions at the various locations by way of example only. This data can be obtained using the transformation techniques described above vis-a-vis transforming the potential data associated with FIGS. 11-14 to the phase realm for FIGS. 17-20. Again by way of example, the data used to develop the potential map based on the data from 64 physical electrodes can be used to develop phase values for 64, 100, 150, 200, 250, 300, 350, 400, 450, 500, 600, 700, 800, 900, 1000, 1250, 1500, 1750, 2000, 2500, 3000, 3500, 4000, 4500, 5000, 6000, 7000, 8000 or more or any value or range of values therebetween in 1 increment locations on the surface (or surface region), and these developed phase values being present for respective time increments at the sampling rate of the monitoring system.

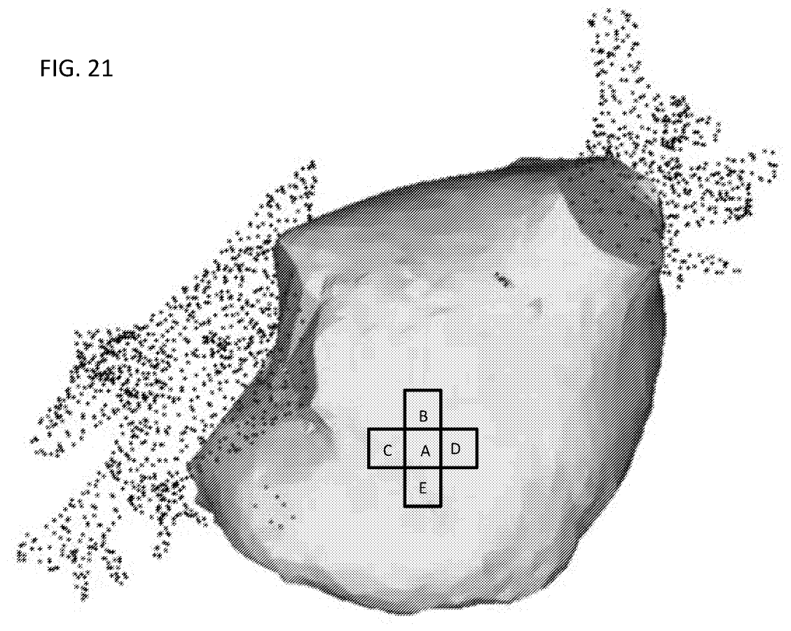

Method 700 further includes method action 750, which includes the action of identifying maximum phase gradients over time for the phase data obtained in method action 740. In this regard, for the various locations on the surface of the chamber, temporally corresponding phase values at the locations are compared to one another. For example, by conceptual explanation only, FIG. 21 shows a posterior view of the left atria with a grid of an exaggerated size superimposed thereon. The grid has five blocks each corresponding to a virtual location of the surface of the chamber, the virtual location corresponding to a location of the phase map (and the potential map). The blocks are labeled A through E for explanation purposes. It is noted that these grids are exaggerated in size and are presented for conceptual purposes only. The concept is that each block represents a location where the inverse mapping actions above developed potential values therefore, and, in method action 740, phase values were developed therefore. Accordingly, for the various temporal locations for which data exists, there are values of phase for locations A, B, C, D and E. In the embodiment of method action 750, for each spatial location the maximum phase difference is retained from the collection of phase differences computed from the difference in phase value from the location and a group of neighboring locations. This computation occurs for each temporal point.

In an exemplary scenario, for explanatory purposes only, with reference to the time increments of FIGS. 17 to 20, for location A, at time 0.0010, respective values for phase of A minus phase of B, phase of A minus phase of C, phase of A minus phase of D and phase of A minus phase of E, are obtained (4 values total). The maximum difference is recorded selected by the greatest absolute magnitude of the four numbers, and this recorded maximum difference could be a positive number or a negative number. Here, for example, the phase between A and C will be the maximum, and it is a positive number. At time 0.5035, for example only by way of presenting this concept, the values are again calculated (they can be calculated for each temporal period in between or portions thereof) and here, the maximum phase is between A and D, and this can be calculated for time 1.005, and the maximum phase difference will be calculated for time 1.500, and so on (the calculations for each block can be done for each measurement cycle, and thus (using the example above) for 13 seconds, there will be 26,000 values for each block in some embodiments). The point is that for each temporal location in at least some exemplary embodiments, there will be a maximum phase gradient for each location relative to locations surrounding that location, and that value will change over time and sometimes that value will be positive and other times that value will be negative, it is also possible for the value to be zero.

The alternating positive/negative value can be because in atrial fibrillation, the electrical impulses are chaotic. Sometimes, the cells at location A will be activated before the cells at location B, because, for example, the electrical wavefront will move generally from South to North (pretending that up is north, down is south, etc.) during some temporal periods, and then the electrical wavefront will move (generally) from north to south in other temporal periods (because of the chaotic nature of atrial fibrillation), and thus the cells at location A will be activated after the cells at location B. And in some temporal periods, the electrical wave front will move from East to West, and thus the tissue cells at location D will be activated before the tissue cells at location A, but the cells at A and B will be activated at approximately the same time. The fronts can (and will) move at various angular directions relative to each location, concomitant with the chaotic nature of atrial fibrillation wavefronts, at least in healthy normal heart tissue.

The point is that the nature of atrial fibrillation will cause the cells to be activated in a random manner relative to one another and thus the phase differences between adjacent locations will change overtime or otherwise be different relative to that which would be the case in a normally functioning heart, where the wavefronts move in direction in a generally consistent manner (e.g., location A will almost always be activated before cell D, if the wavefront moves from the Northwest to the south East, etc.).

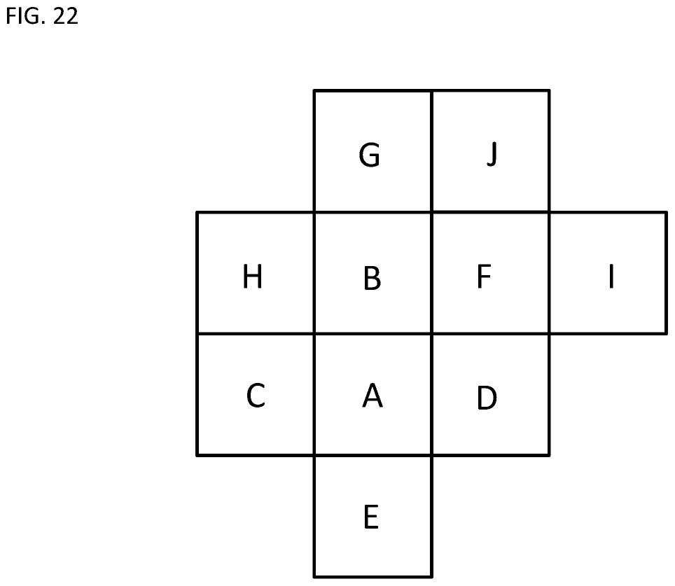

FIG. 22 presents an expanded grid system to conceptually explain the concept under explanation in some greater detail. In the exemplary embodiment where phase gradients are being calculated for each temporal location (or the desired temporal locations, which can be a subset of the total number of data collection cycles), there will also be phase difference calculations for location B, and thus there will be a phase difference calculation for B vs. G, B vs. F, B vs. A and B vs. H, and for location F, phase differences calculated at F vs. J, F vs. I, F vs. D, F vs, B, and so on. For each temporal location (in an embodiment), the maximum phase difference at each location is determined. For time 0.5000 for example, the maximum gradient for A could be with location E, and the maximum gradient for B could be with F, for example. These values are recorded for time 0.5000. And note that such values should exist for each location (A, B, C, D, E, F, G, H, I, J and not shown locations K, L, M and so on—again, embodiments can include thousands of locations) for each temporal period. Accordingly, method action 750 can result in tens of thousands of values for the maximum phase difference for each location over a 10 or 15 seconds of time period by way of example.

It is briefly noted that in the embodiment under description, only the four 90 degree adjacent locations (nodes or points) of the phase map are utilized. This is for computational convenience. In some embodiments, the diagonal nodes could be utilized in addition to this or instead of the 90° adjacent locations. In some other embodiments, some other form of comparison selection regime can be utilized, such as, for example, all locations within X millimeters of a given location. In some other embodiments, the set of neighboring locations could be fewer than 4. Any set of locations the phase difference of which can be calculated that can enable the teachings detailed herein to be implemented in the utilitarian value can be utilized in at least some exemplary embodiments.

In any event, upon completion of method action 750, there will be resulting data that includes the maximum phase gradients over time for the phase data obtained in method action 740 for each location or however many locations that are utilitarian. This then leads to method action 760, which includes executing time averaging to obtain regional atrial fibrillation fingerprints. In this regard, for a given location, the many maximum phase gradient values obtained in method action 750 are time averaged and resulting value for each location are obtained.

In an embodiment, it can be sufficient to simply add the values of the maximum gradients together and evaluate the result. The normal tissue will result in a value of zero or close to zero. The tissue that is a driver and/or a substrate of afib will result in a non-zero value in a statistically significant manner. And of course there are other methods of evaluating the phase gradients to determine whether or not a given location is experiencing a nonrandom activation. In an exemplary embodiment, artificial intelligence or a neural network or a deep neural network or otherwise a trained neural network can be utilized to analyze the phase gradient data to identify the locations that are the drivers of afib/substrates of afib.

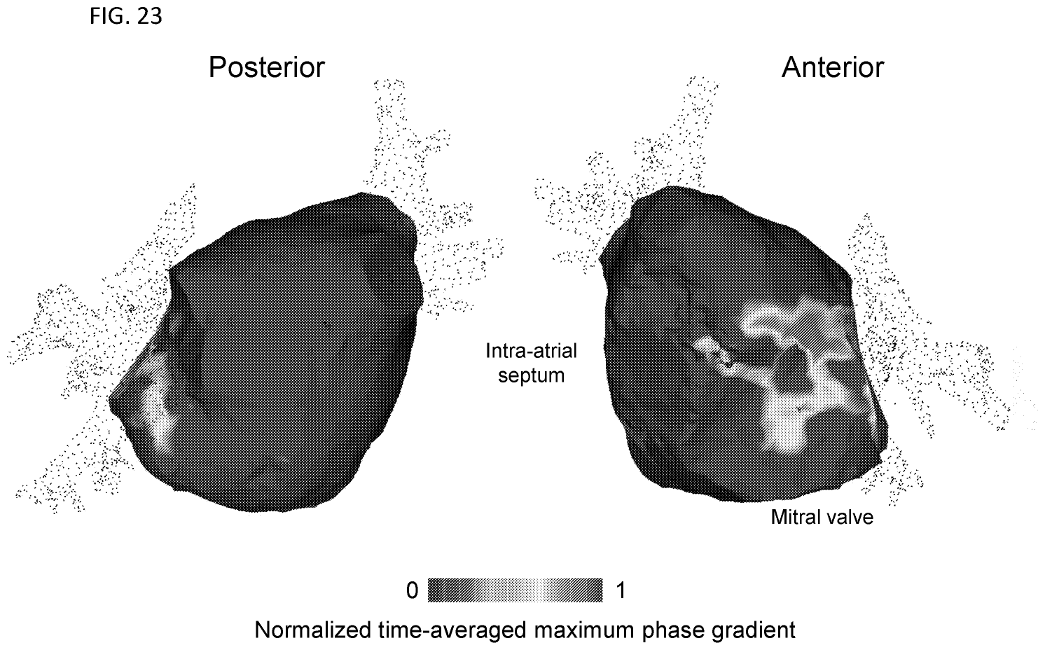

FIG. 23 visually presents the results of the action of time averaging the maximum phase gradients at the various locations on the surface of the chamber. Here, it shows that for the vast majority of locations on the surface of the chamber, the normalized time average maximum phase gradient is zero or close to zero (note that normalization is used for convenience-embodiments may not normalize the data). That is, for the various thousands of maximum phase gradients at each location, over time, the maximum gradients cancel out each other. This is because at some temporal periods they are positive and at other temporal periods they are negative, and so on, because of the chaotic nature of the wavefronts in a heart afflicted by atrial fibrillation. But inspection of the features of FIG. 23 reveal that there are some areas on the surface of the chamber where the normalized time average maximum phase gradient is not zero. This is because the tissue at those locations is interfering in some consistent manner with the chaotic wavefronts. Put another way, there is something that is interfering with the randomness of the activation of the cells in these locations relative to that which would otherwise be the case. In at least some embodiments, the tissue that is interfering with the randomness is tissue that is exasperating or otherwise causing the symptoms of the atrial fibrillation in the first instance. This tissue can be a driver and/or a substrate of afib.

More specifically, the teachings herein enable the testing of whether phase signatures do repeat in specific locations more regularly than would be expected for normal heart tissue subjected to afib. This is done by averaging maximum phase gradients over time to create a phase signature map. FIG. 23 identifies the phase signature map for a specific patient at the current state of their disease condition based on maximum phase gradients (derived from electrical activation wave fronts) for all regions over the recording period of the procedure. The averaging executed per region over times from 5-35 seconds for example (or longer or shorter or any time that provides utilitarian values that enable the teachings herein—the time is typically the time used to collect the potentials using the electrodes). The resultant maps show that activation, in this atrial fibrillation scenario, was preferentially initiated in antero-lateral region (red and yellow) just below the LA roof and spreading toward the mitral valve, and that activation occurs with equal probability everywhere else (blue). Using other measures, one can identify other atrial fibrillation fingerprints. Time-averaged spatial phase gradient maps indicate regions in which electrical activation is repeatedly delayed with respect to adjacent tissue, while time-averaged entropy maps identify regions with complex electrical activity.

It is briefly noted that in a normal heart without afib, the normalized time averaged maximum phase gradient over the chamber may not be blue or otherwise may not have time average to zero. This is because there will always be a phase difference between one location and at least one other location adjacent that location when wavefronts move consistently, and thus there is always a maximum phase difference with respect to a given location relative to at least one other location. The maximum phase differences cancel out owing to the randomness of afib, and that is the fact upon which at least some of the teachings herein rely. Conversely, instead of the ordered wave progression, activating heart cells as the wave progresses, in scenarios where the atrial fibrillation scenario causes activation of the cell, the activations will be random and thus the phase difference between adjacent cells will cancel each other out over time, at least for cells that do not influence the afib or are otherwise healthy or normal.

And the maps are highly repeatable as the time interval increased from 5 to 35 sec, can be co-located and internally consistent and observed in different locations across patients. Using method 700, one can identify the substrates that drive afib and provide appropriate targets for isolation (e.g., via ablation). Embodiments include ablating those areas or some of those areas or otherwise areas relative to or based on those areas, or otherwise providing instructions to do so.

Because the atrial fibrillation fingerprints are stable over time, embodiments include building the data over the surfaces of both atria by sequential region-of-interest mapping (e.g., by incrementally moving the catheter to different regions in the chamber, thus enabling the basket to be used in a state where the electrodes are closer to each other than that which would otherwise be the case. Moreover, it is noted that embodiments include obtaining the potential data with electrodes no more than 5, 6, 7, 8, 9, 10, 11, 12, 13, 14, 15, 16, 17, 18, 19, 20, 21, 22, 23, 24, 25, 26, 27, 28, 29 or 30 mm or any value or range of values therebetween in 1 mm increments from the adjacent surface of the chamber.

In view the above, it can be seen that in at least some exemplary embodiments, there is a method, comprising obtaining heart phase data for a plurality of activation cycles of a living human afflicted with atrial fibrillation and analyzing the heart phase data to identify specific heart tissue locations where there are repeated and consistent temporal discrepancies of electrical activation relative to other tissue locations. In an exemplary embodiment, the action of obtaining the heart phase data can be executed by executing one or more of the actions detailed herein, such as by implementing a Hilbert transformation, or by using other numerical techniques or by pure number crunching is such can be utilitarian. In at least some exemplary embodiments. The action of obtaining heart phase data can be executed by receiving from a remote processor or the like pre-calculated heart phase data. The action of identifying the specific heart tissue locations with our repeated and consistent temporal discrepancies can be executed according to anyone where the teachings detailed above. In an exemplary embodiment, this method can be executed for any one or more of the values of location herein for any one or more of the data collection numbers frequencies and/or for any one or more of the temporal periods detailed herein whether method action of analyzing the heart phase data is executed in a period of time within 10, 15, 20, 25, 30, 35, 40, 45, 50, 55, 60, 70, 80, 90, 100, 120, 130, 140 or 150% or any value or range of guys therebetween in 1% increments of the temporal period that form the basis of the data collection. For example, if the data was collected over 10 seconds, the method action of analyzing the heart phase data can be executed within three seconds or 15 seconds.

In an exemplary embodiment, the action of analyzing the heart phase data includes implementing a statistical analysis on the heart phase data. By way of example only and not by way limitation, this can correspond to the time averaging detailed above or otherwise implementing signal averaging techniques. Thus, the action of analyzing the heart phase data can include implementing time averaging analysis on the heart phase data. In an exemplary embodiment, the utilization of the time averaging permits the values to become stable within a very short period of time, such as any one or more the aforementioned times detailed above. Time averaging supports the generation of a fingerprint image which becomes static after a few seconds of data. Without time averaging, in some embodiments, the phase map is continuously changing and the clinician is trying to identify areas which are not changing. Time averaging reduces the highly variable and active areas down to zero which shows the non-zero areas which are areas of interest, in some embodiments.

In an embodiment the action of analyzing the heart phase data includes, for a plurality of spatial locations on an interior surface of the heart, which spatial locations include the identified specific heart tissue locations, identifying respective maximum phase gradients for respective locations of the plurality of spatial locations over a length of time, time averaging the respective maximum phase gradients for the respective locations an identifying corresponding locations where the time averaged results are statistically aberrant and/or are not statistically aberrant, wherein the identified corresponding locations of the time average results that are statistically aberrant are the identified specific heart tissue locations and/or the identified corresponding locations of the time average results that are not statistically aberrant are not the identified specific heart tissue locations. By way of example only and not by way of limitation, in an exemplary embodiment, the time average results that are statistically aberrant can be those that are substantially nonzero. By way of example only and not by way of limitation, in an exemplary embodiment, the time average results that are statistically aberrant can be those that are different from the majority of values. By way of example only and not by way limitation, in most parts, most of the tissue will be normal, at least in most parts they can be treated. In this regard, instead of operating on a binary time average zero versus time average nonzero, a more flexible approach can be utilized that statistically analyzes the entire data set for all of the locations or at least most of the locations, and then determines which of the locations indicate statistically aberrant time values or otherwise time averaged values.

Corollary to this is that the time averaged results that are not statistically aberrant could be the majority of the results or otherwise could be the results that are closer to zero then a subset of other results. Statistical analysis can be implemented to identify the non-aberrant time averages. In some embodiments, the knowledge of one of or new skill in the art can be relied upon to identify the values that are statistically aberrant and/or not statistically aberrant. Indeed, the requirement that there be statistical aberrant and/or non-aberrance can be dispensed with in some embodiments. In this regard, a nonstatistical approach could be applied based on underlying knowledge.

In an embodiment, the action of analyzing the heart phase data includes, for a plurality of spatial locations on an interior surface of the heart, which spatial locations include the identified specific heart tissue locations, identifying respective maximum phase gradients for respective locations of the plurality of spatial locations over a length of time, time averaging the respective maximum phase gradients for the respective locations and identifying corresponding locations where the time averaged results are non-zero and/or statistically zero, wherein the identified corresponding locations of the time average results that are non-zero are the identified specific heart tissue locations and/or the identified corresponding locations of the time average results that are statistically zero are not the identified specific heart tissue locations

In an embodiment, the respective maximum phase gradients are the respective maximum phase gradients between the respective locations and a plurality (2, 3, 4, 5, 6, 7, 8, 9, 10, 11, 12, 13, 14, 15 or more or any value or range of values therebetween in 1 increment) of proximate locations on the surface of the heart.

In an embodiment, the proximate locations are effectively North-South-East-West adjacent locations and/or can be the four closest nodes. The locations can be the 2, 3, 4, 5, 6, 7, 8, 9, 10, 11, 12, 13, 14, 15 or more or any value or range of values therebetween in 1 increment closest nodes. The locations can be the locations immediately surrounding the respective location. The locations can be the locations that provide utilitarian value with respect to implementing the teachings herein.

Some embodiments include obtaining respective plurality of temporally spaced electrical potentials for respective electrodes of at least 20, 30, 40, 50, 60, 70, 80, 90, 100, 120, 150, 200, 250, 300, 350, 400, 450 or 500 or more electrodes of a catheter located in a heart chamber at a first location in the heart chamber, converting the obtained respective plurality of temporally spaced electrical potentials to the heart phase data, thereby obtaining the heart phase data. In these embodiments, the actions of identifying respective maximum phase gradients, time averaging, identified corresponding locations where the time average results are statistically aberrant and/or not statistically aberrant, are based on the obtained respective plurality of temporally spaced electrical potentials for the catheter located at the first location.

In some embodiments, the method includes (with respect to repositioning the catheter to map another different region) obtaining respective second plurality of temporally spaced electrical potentials for respective electrodes of at least 20, 30, 40, 50, 60, 70, 80, 90, 100, 120, 150, 200, 250, 300, 350, 400, 450 or 500 or more or any value or range of values therebetween in 1 increment electrodes of the catheter located in the heart chamber at a second location in the heart chamber different from the first chamber, converting the obtained respective second plurality of temporally spaced electrical potentials to second heart phase data, for a plurality of second spatial locations on the interior surface of the heart, which second spatial locations include the identified specific heart tissue locations, identifying respective second maximum phase gradients for respective second locations of the plurality of second spatial locations over second length of time, second time averaging the second respective maximum phase gradients for the second respective locations; and identifying corresponding second locations where the time averaged second results are statistically aberrant and/or not statistical aberrant, wherein the identified corresponding second locations of the second time average results that are statistically aberrant are included in the identified specific heart tissue locations and/or the identified corresponding second locations of the time second average results that are not statistically aberrant are not included in the identified specific heart tissue locations.

In an embodiment, this obtaining of the temporally spaced electrical potentials can occur for a third plurality of temporally spaced electrical potentials, and 4^{th} plurality and a 5^{th} plurality and so on for each of the locations where the catheter is moved to within the chamber, and the associated method actions there with can be repeated accordingly. The results can be combined to obtain a phase map or otherwise to evaluate the entire surface array much larger portion of the surface relative to that which exists with respect to the original temporally spaced electrical potentials that were obtained prior to the action of obtaining the respective second plurality of temporally spaced electrical potentials.

In an embodiment, the action of obtaining heart phase data and analyzing is executed in real time vis-à-vis a catheter located in a heart chamber.

In an embodiment, the action of obtaining heart phase data and analyzing is executed while the catheter is located in a heart chamber. In am embodiment, these actions are executed while the patient is in the reading room or otherwise before the patient leave the operating room in which the potentials were obtained from the catheter in the patient. In an exemplary embodiment, these actions are executed within one, two, three, four, five, six, seven, eight, nine, 10, 11, 12, 13, 14, 13, 14, 15, 16, 17, 18, 19, 20, 21, 22, 23, 24, 25, 26, 27 28, 29, 30, 35, 40, 45, 50, 55, 60, 65, 70, 75 or 80 or 90, or 100, or 110 or 120 minutes or any value or range of values therebetween in one minute increments from the beginning and or middle and/or an end of any one or more of the above-noted or herein noted medical procedure(s) that is/are implemented on the heart of the patient (e.g., ablation) based on the results of the action of analyzing.

Indeed, in an embodiment, the action of sensing the potentials with the electrodes, the actions of executing method 700 for example or the actions of analyzing, and the action of ablating to completion are executed within some of the above noted periods (which are not repeated here for purposes of textual economy).

In an exemplary embodiment there is a method that includes the action of developing a time-varying electrical potential map of a surface of a cavity of a beating heart. This action can be accomplished by one or more of the methods otherwise the sub-methods detailed above vis-à-vis transforming the electrical potentials obtained from the electrodes to the surface on the heart chamber. By way of example only and not by way of limitation, this can be done by the inverse mapping method detailed above, using, for example, a meshless method. Any one or more of the method actions detailed in the above referenced '112 patent can be executed to execute this action.

This method further includes the action of developing a time-varying phase map of the surface of the cavity based on the developed time-varying electrical potential map. The techniques associated with developing the time varying phase map according to some embodiments are detailed above. Any other technique that can result in a time varying phase map, or a data equivalent thereof, can be used providing that such enables the teachings detailed herein. And it is noted that the phase map need not have one to one correspondence with the potential map. For example, the potential map may have 1500 (by way of example only) locations for which respective potentials that are developed on a time varying basis. The developed phase map could have fewer locations by way of example only. It can be utilitarian to only analyze some locations or otherwise a subset of the locations, and thus only some of the phase data will be developed for only some of the locations. This could save computational time or otherwise be utilitarian with respect to eliminating extraneous locations based on the results of the creation of the potential map. The elimination of the extraneous locations would thus provide more accurate results with respect to the phase transformation or otherwise with respect to the data to be extracted from this phase transformation. In an embodiment, at least 50, 55, 60, 65, 70, 75, 80, 85, 90, 91, 92, 93, 94, 95, 96, 97, 98, 99 or 100% or any value or range of values therebetween in 1% increments of the locations are subject to the transformation from the potential realm to the phase realm.

The method under discussion can further include the action of identifying repeating phase signatures for respective locations on the surface of the atrial cavity from the time-varying phase map that repeat in a statistically aberrant manner relative to other phase signatures at other respective locations. As detailed, embodiment, by implementing time averaging of the maximum phase gradients at the various locations over various temporal periods detailed herein, or any other that can have utilitarian value, signatures that have aberrant occurrences can be identified.

In an embodiment, the electrical potential map has at least 100, 200, 300, 400, 500, 600, 700, 800, 900, 1000, 1250, 1500, 2000, 3000, 4000, 5000 or more or any value or range of values therebetween in 1 increment electrical potential spatial locations and at least respective 500, 750, 1000, 1500, 2000, 2500, 3000, 3500, 4000, 4500, 5000, 6000, 7000, 8000, 9000, 10000, 12K, 13K, 14K 15K 16K 17K 18K 19K, 20K, 25K, 30K, 35K or 40K or any value or range of values therebetween in 1 increment temporal potential values for the respective potential spatial locations, the phase map has at least any of the just noted values for the potential map phase spatial locations and the values can be different (we use the reference for textual economy) and at least the aforementioned number for the potential temporal values temporal phase values (and again they may not be the same-we reference for textual economy) for respective phase locations. In some embodiments, the respective electrical potential locations of the specified number of electrical potential locations have respective phase locations of the at least specified number of phase locations, but again, the number need not be the same (but can be). In some embodiments, the values are within 1, 2, 3, 4, 5, 6, 7, 8, 9, 10, 11, 12, 13, 14, 15, 16, 17, 18, 19, 20, 21, 22, 23, 24 or 25% or any value or range of values therebetween in 1% increments of the lower value (and this can be the case for the temporal locations as well).

In some embodiments, the actions of developing a time-varying electrical potential map, developing the time-varying phase map, and identifying the repeating phase signatures are executed within a period of no more than 30, 25, 20, 15, 10, 9, 8, 7, 6, 5, 4, 3, 2 or 1 minutes. Indeed, in some embodiments, the time averaging phase data reaches convergence within 40, 35, 30, 25, 20, 15, 10, 9, 8, 7, 6, 5, 4, 3 or 2 seconds of the beginning of the calculations of the maximum phase gradients. (Convergence can mean that the statistically non-aberrant numbers (e.g., the zero values) average out to a level number.

In some embodiments, the action of developing a time-varying electrical potential map of a surface of a cavity of a beating heart is based at least in part on time-varying readings from electrodes located in the cavity, and the action of identifying the repeating phase signatures is executed within 10, 9, 8, 7, 6, 5, 4, 3, 2 or 1 minutes or any value or range of values therebetween in 1 minute increments of the electrodes being removed from the chamber (and this covers the electrodes being in the chamber).

In some embodiments, the action of developing a time-varying electrical potential map of a surface of a cavity of a beating heart is based at least in part on invasive readings taken while a human in which the beating heart resides is in an operating room/before he or she leaves the OR, and the action of identifying the repeating phase signatures is executed while he or she is in the OR/before the human leaves the operating room.

In some embodiments, the method include executing a medical procedure targeted at tissue of the heart corresponding to at least some of the respective locations identified as having the repeating phase signatures that repeat in the statistically aberrant manner before the human leaves the operating room/within 20, 19, 18, 17, 16, 15, 14, 13, 12, 11, 10, 9, 8, 7, 6, 5, 4, 3 or 2 minutes of the catheter first entering the cavity.

In an embodiment, the method includes, after executing the medical procedure, repeating any one or more of the method actions detailed herein, to “validate” or determine whether or not the procedure was effective in whole or in part. For example, after ablating the tissues that were identified as having the aberrant activation times or otherwise ablating tissue based on the locations identified as having aberrant activation times, additional electrode readings are taken, which readings can be taken along any of the lines detailed above or differently providing such is utilitarian, and then the potential map is re-created utilizing these new readings, and in the phase map is re-created utilizing these new phase maps, and then the statistical analysis is reexecuted or another type of analysis is executed on the phase map, or more accurately, on the maximum phase gradients for the various locations, and locations with aberrant time average values or locations without aberrant time average times are identified, and compared to the original set of such locations. This new data is used to validate the efficacy of the medical treatment. If there are remaining tissue locations with aberrant readings, a second treatment can be executed, and then a new set of test can be run, and so on. All of this can be done while the patient is in the OR/before he or she leaves the OR, and this can be done within any of the total time periods detailed herein for such procedures providing that the art enables such.

Thus, there is an exemplary method of developing second time-varying electrical potential map of the surface of the cavity of the beating heart, developing second time-varying phase map of the surface of the cavity based on the second developed time-varying electrical potential map and evaluating whether and/or how many repeating phase signatures for respective locations on the surface of the atrial cavity from the second time-varying phase map that repeat in a statistically aberrant manner relative to other phase signatures at other respective locations, and based on the evaluation, evaluation whether the medical procedure was successful, etc.

In an embodiment, there is a method, comprising developing data including at least X spatial locations and at least Y respective phase gradients for the respective spatial locations of the X spatial locations, analyzing the developed data (according to any of the regimes herein), which analysis can be executed by statistical analysis or any other analysis that will provide utilitarian value, and identifying locations of the respective locations that are indicative of tissue influencing atrial fibrillation based on the statistical analysis, wherein X is at least any of the spatial values herein and Y is at least any of the temporal values herein. In an embodiment, the analysis is time averaging.

In an embodiment, the analysis can entail adding all the phase gradients at each location and looking for resulting values that are aberrant. Indeed, even without time averaging, if indeed values will add to zero for the non aberrant tissue, the numerator will likely be zero in some embodiments, so the averaging will be the averaging of zero.

In some embodiments, the action of identifying locations includes identifying locations where the statistical analysis of the developed data indicates non-random activation of respective heart tissue cells at the identified locations. In some embodiments, the action of identifying locations includes identifying locations where averaging of the maximum phase gradients yields a statistically meaningful non-zero value. In some embodiments, the action of identifying locations includes identifying locations where averaging of the maximum phase gradients yields a value that is at least 0.2, 0.3, 0.4, 0.5, 0.6, 0.7, 0.8, 0.9, 1, 1.1, 1.2, 1.3, 1.4, 1.5 or 1.6 or more standard deviations from the mean average for all of the average phase gradients of the locations (these can be the aberrant locations). In some embodiments, the action of identifying locations includes identifying locations where averaging of the maximum phase gradients yields a value that is no more than 0.2, 0.3, 0.4, 0.5, 0.6, 0.7, 0.8, 0.9, 1, 1.1, 1.2, 1.3, 1.4, 1.5 or 1.6 or more standard deviations from the mean average for all of the average phase gradients of the locations (these can be the non-aberrant locations). And with respect to these below standard deviation values, this can encompass time averages where the time average is not zero out for the healthier normal tissue, for whatever reason, based on, for example, the physiology of a given individual.

In some embodiments, the action of identifying locations includes identifying other locations where averaging of the maximum phase gradients of at least a majority of the Y phase gradients yields a statistically zero value. In some embodiments, the action of identifying locations includes identifying locations where averaging of the maximum phase gradients of at least a majority of the Y phase gradients yields a statistically meaningful non-zero value and the action of identifying the locations includes further statistically analyzing the values of the non-zero values. Here, this can utilize to further vet the non zero values. For example, there could be statistically significant nonzero values that are still indicative of healthy tissue or tissue that is not a substrate for afib, or at least not a meaningful influencer of afib. These might not be targets for ablation, at least not at an initial pass. For example, only some of the locations that have the statistically significant nonzero value might be ablated, such as those that are above average of all of the locations that are statistically significantly nonzero, and then the potential data measurements could be re-taken and then the phase analysis could be rerun to see the new activation patterns, and then a percentage or some threshold amount of that tissue (e.g., the above average for the new non-zero locations) could be ablated, and so on. Point here is that by further analyzing the statistically significant nonzero locations, additional choices can be made as to what tissue should be targeted or not targeted.

In some embodiments, the statistical analysis of the developed data identifies statistically consistent patterns of electrical activity that repeat in a statistically meaningful manner over time.

As noted herein, embodiments, include non-transitory computer readable medium having recorded thereon, a computer program for executing at least a portion of a method, the computer program including any one or more all the actions detailed herein providing that the art enable such. In an exemplary embodiment, the computer readable medium includes code for statistically analyzing first data based on phase gradients for at least X locations (where X can be any of those detailed herein) on a surface of a chamber of a human heart and code for identifying a plurality of locations from the at least X locations, based on the statistical analysis of the first data, that should be targeted for treatment. In an embodiment, the medium has code for transforming respective electrograms for respective locations of the at least X locations to a phase record including the phase gradients. There can be code for creating the electrograms from data based on electrical potentials obtained from electrodes within the human heart, the number of electrodes within the human heart being less than X, such as at least 10, 20, 30, 40, 50, 60, 70, 80, 85, 86, 87, 88, 89, 909, 91, 92, 93, 94, 95, 96, 97, 98 or 99% or more or any value or range of values in 1% increments less than X. The code for creating the electrograms uses inverse solution methods. In an embodiment, the code for statistically analyzing the first data time averages respective maximum phase gradients for the at least X locations. In an embodiment, the code for statistically analyzing the first data time averages respective maximum phase gradients for respective locations of the at least X locations and the code for identifying the plurality of locations from the at least 150 location identifies respective locations where time averages of the respective maximum phase gradients are statistically significantly non-zero.

In an embodiment, the at least X locations includes at least 2,000 locations, respective locations of the at least 2,000 locations have at least 1000 respective maximum phase gradients and the medium creates the electrograms and identifies the plurality of locations from the at least 2,000 locations for the at least 1000 respective maximum phase gradients within 20, 15, 10, 9, 8, 7, 6, 5, 4, 3, 2 or 1 minutes when run on a Dell™ laptop with an Intel Core i9 Microprocessor with at least a 2.8 GHz clock frequency, at least 16 by 1024 KB L2 cache, at least 22.00 MB L3 cash, a TDP of at least 160 W, a DMI 3.0 I/O bus and a 4×DDR4-2666 memory and in some embodiments, the code for creating the electrograms uses at least 500 measurements from each electrode per second.

FIG. 10 shows a diagram of an exemplary system that can implement one or more of the method actions herein. A catheter is placed inside a volume of interest, typically a heart chamber. Catheters are electrically connected to an interface 13, which is electrically isolated and may comprise a proprietary system or a set of such systems. Instantaneous potentials and the 3D positions are acquired from individual electrodes on one or more cardiac catheters. For instance potentials and 3D positions may be recorded simultaneously from multi-electrode basket catheters positioned in the RA and LA, or from a multi-electrode basket catheter and an ablation catheter in the same cardiac chamber. 3D electrode positions are recorded using impedance techniques, magnetic sensors, ultrasound sensors or combinations of these methods. Electrocardiograms (ECGs) are also acquired without position information for standard lead configurations.

The processing unit 14 controls the acquisition and processing of data so that recorded potentials or information derived from them can be mapped onto the endocardial surface of a heart chamber or chambers in a form that is useful to the operator. The processing unit is an electronic computing device with a real-time operating system and it may include a field programmable gate array (FPGA) and appropriate memory. It may be connected to the interface 13 either directly or wirelessly.

Alternative forms of the teachings herein can comprise a computer program or computer readable medium adapted to perform the method. The processor unit or means may comprise a microprocessor, FPGA, logic circuit or other form of processor. The processor means may be incorporated into a computer, for example a PC, mainframe, remote server or cloud-cluster. The processor means may be a single device or may interface with further processing means. Connections may be made between the processing means through a network or wired/wireless communications. The processor or processing means may receive information from a data storage device such as a removable storage disk, hard-disk, ROM, RAM or a network connection.

The first processing step is to construct a computer representation of the 3D endocardial surface geometry of the heart chamber or chambers of interest. This may be derived from i) cardiac MR images ii) contrast-enhanced cardiac CT images or iii) surface coordinates mapped under fluoroscopic guidance using a contact catheter. Alternately, geometry created in iii) can be merged with endocardial surfaces segmented from i) or ii). Static 3D models can be integrated with cine-fluoroscopic imaging or ultrasound imaging to provide estimates of heart wall motion. Provision for the import of such video data is indicated in 15.

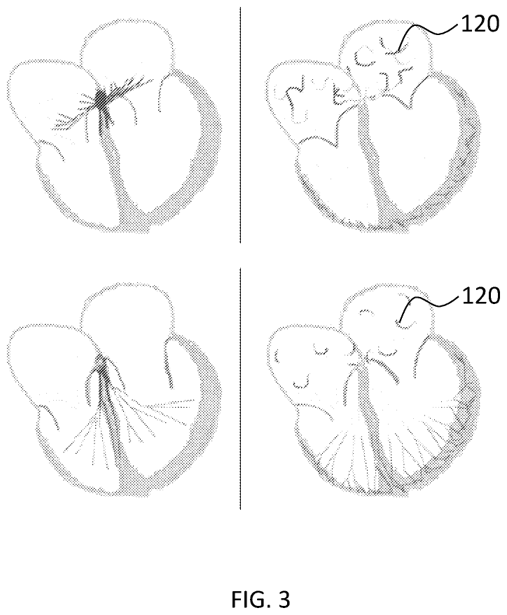

Processing steps that will be carried out during the acquisition of data from a catheter or catheters include those described in relation to FIGS. 3, 4 and 5 above. For each time interval, a forward solution will be used to estimate potentials inside the catheter followed by an inverse solution that enables potentials to mapped onto the endocardial surface of the cardiac chamber or chambers. This will be repeated at successive intervals throughout a recorded data set.

Endocardial potentials will be rendered on a computer representation of the 3D surface of the heart chamber or chambers presented on a screen or display device 16 in a form that can be manipulated interactively by the operator. The location of catheter or catheters with respect to the heart wall will also be displayed. Electrograms at selected endocardial points (or at selected catheter electrodes) and selected ECG leads will be presented simultaneously in a moving window with an adjustable time base. It will also be possible to display a projection of unique source points onto the endocardial surface providing intuitive information on the spatial resolution of the inverse maps presented. The processing and display of time-varying endocardial potentials can be completed in real-time or near real-time. These may include the following:
spatial and temporal filtering
activation time analysis
regional variability analysis
regional frequency analysis
phase analysis
   The results of the analysis steps listed will be mapped onto a representation of the 3D heart displayed in 11.

The processing steps may be implemented in hardware or software or a combination of these. The steps or instructions may be stored in computer readable media or memory including a hard disk, random access memory (RAM), read only memory (ROM) a removable storage disk or device or other storage media.

There are methods of operating a catheter in a sequence of steps guided by the information displayed in 11 above. Initially a global picture of electrical activity on the endocardial surface of a heart chamber will be acquired and displayed. This can use a catheter positioned in a first position, generally centrally with electrodes in contact with or adjacent to as much of the endocardial surface of the heart chamber as possible. The catheter may be moved to one or more second positions to investigate regions of interest. As noted above, a catheter can be used for global mapping can also be used for regional mapping, such as by reducing the dimensions of the catheter to move the electrodes closer to each other to obtain more accurate readings, for example, by adjusting the size of the catheter to map in specific regions of the chamber with greater precision.

In a method of mapping of electrical activity, the activity is obtained over a short period of time (for instance continuous periods of at least 3 to 50, or 5 to 40 seconds are required in AF) before a user decides which areas require further investigation. Higher resolution mappings will be obtained in these regions-of-interest by moving multi-electrode arrays with smaller diameters into them (again in AF continuous periods of at least 10-20 seconds are required for region-of-interest mapping). This method will support more efficient high-resolution endocardial mapping of electrical activity because it utilizes potentials recorded at all electrodes whether they are in contact with the endocardial surface of the heart chamber or not. The operator will also receive direct feedback the accuracy of endocardial maps through visual comparison of maps and electrograms displayed as the catheter is moved closer to the surface and as some electrodes make contact with it.

The mapping approach above could be carried out using combinations of catheters with different dimensions. In some embodiments, a single adjustable catheter can be used. Here the dimensions of the electrode array are altered by withdrawing or advancing the splines into or out of the catheter. In some embodiments, the catheter can meet the following general specifications:
The catheter will be steerable.
It will be possible to lock the dimensions of the electrode array in multiple dimensions between fully open and fully closed states.
Electrodes will be uniformly spaced as far as is possible in open and closed states and distributed evenly across the mathematically closed virtual surface that bounds them.
Inter-electrode spacing will be sufficient to characterize electrical activity appropriately within endocardial regions on the order of 10 mm diameter.

It can be possible to introduce the catheter into atrial appendages and pulmonary vein in a closed state.

In summary combination of the processes detailed herein can provide a regime for mapping endocardial electrical activity on a heart chamber or chambers. This is achieved, in part, by mapping potentials recorded on a catheter containing multiple electrodes back to the endocardial surface when some or all of the electrodes are not in contact with it. This can involve the solution of an inverse problem that is inherently ill-posed. This can involve executing a solution of the forward problem inside the catheter, which provides additional information that improves the conditioning of the inverse problem enabling more robust solutions, independent of the method used. Inverse mapping can be used to reconstruct global endocardial potentials. However, local or region or interest (regional) mapping can be executed to fully reconstruct endocardial surface potentials in regions where endocardial geometry is complex and/or there is rapid spatial variation of endocardial potentials. Meshless maps can be used to reduce the scale of the inverse problem enabling acceptably rapid solution in the presence of relative motion of the electrodes and the heart wall. By combining appropriate systems for acquisition, processing and display of endocardial mapping data (see FIG. 10), with improved multi-electrode catheters, it is possible to provide more accurate, more rapid and more comprehensive results than with existing methods.

From the foregoing it will be seen that a method of determining physiological information of an endocardial surface is provided which improves the calculation of physiological information, for example, electrical potentials.

Embodiments include executing treatment methods based on the obtained data with respect to the locations identified as being aberrant or otherwise having delayed phase gradients were otherwise based on the results of method 700. In an exemplary embodiment, the method includes ablating some or all of the tissue at the locations having the non-zero gradients otherwise having the time averaged regional afib fingerprints. In some exemplary embodiments, a level of the time averaged maximum phase gradient is set, above which the treatment method is executed to the tissue associated there with. With reference to FIG. 23 and the normalized time average maximum phase gradient map shown in that figure, tissue corresponding to a maximum phase gradient of above 0.65 by way of example could be targeted for ablation. In an embodiment, this can be a hard number based on empirical results over a statistically significant number of patients, while in other embodiments, this can be based on the general overall impression of a given result for a given patient. With respect to FIG. 23, it can be utilitarian to only ablate the area in red or around the red area and not treat the green areas even though the green areas may be as extensive as the red areas, but the green areas are not as intense as the red areas with respect to the time averaged maximum phase gradient values and thus it may be sufficient to only “process” the tissue that shows up as red. Conversely, the extensive nature of the areas in green with respect to spatial location could be tissue that serves as a greater block to the electrical signals then the tissue in red. In any event, methods include evaluating the data obtained from method 700 or whatever method is derived from the teachings detailed herein to obtain the data related to heart tissue that has statistically significant aberrant activation times and, based on that data, treating a portion of the heart via a medical procedure, such as for example, ablation therapy.

In an exemplary embodiment, the medical therapy is executed to competition within GHI minutes from the time that method 700 or any other related method relating to evaluating the time average maximum phase gradient or any other data that is utilitarian relating to identifying the tissue that is aberrant is completed, where GHI is 10, 15, 20, 25, 30, 35, 40, 45, 50, 55, 60, 65, 70, 75, 80, 85 or 90 or any value or range of values there between in 1 increment. In an exemplary embodiment, the medical therapy is executed to completion within GHI minutes from the time that method 700 or any other related method relating to evaluating the time average maximum phase gradient or any other data that is utilitarian relating to identifying the tissue that is aberrant is first begun, and this need not be the same as the aforementioned period. In an exemplary embodiment, the medical therapy is executed to completion within GHI minutes from the time that the catheter first enters the chamber, and this need not be the same as the aforementioned period.

In an embodiment, method 700 or a truncated method thereof is executed to completion within JKL minutes from the time that method 700 is commenced, where JKL can be 2, 3, 4, 5, 6, 7, 8, 9, 10, 15, 20, 25, 30, 35, 40, 45, 50, 55 or 60 or any value or range of values there between in 1 increment. In an embodiment, method 700 or a truncated method thereof is executed to completion within JKL minutes from the time that the catheter first enters the heart chamber. And this time need not be the same as the prior period. And method 700 can be executed to cover one or more regions within the chamber, and can be implemented for any of the location values detailed herein at any of the cycle values for any of the time periods detailed herein.

Any control unit and/or test unit or the like disclosed herein can be a personal computer programs was to execute one or more or all of the functionalities associated there with are the other functionalities disclosed herein. In an exemplary embodiment, any control unit and/or test unit or the like can be a dedicated circuit assembly configured so as to execute one or more or all of the functionalities associated there with or the other functionalities disclosed therein. In an exemplary embodiment, and the control unit and/or test unit or the like disclosed herein can be a processor or the like or otherwise can be a programmed processor. The control unit can be a signal processor or the like or a personal computer or the like or a mainframe computer or the like etc., that is configured to receive signals from the electrodes or data based on data from the electrodes and implement method 700 for example or a related action. More particularly, the control unit can be configured with software the like to analyze the signals/the data based on the signals in real time and/or in near real time while the catheter is in the chamber.

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

An exemplary system includes an exemplary device/devices that can enable the teachings detailed herein, which in at least some embodiments can utilize automation, as will now be described in the context of an automated system. That is, an exemplary embodiment includes executing one or more or all of the methods detailed herein and variations thereof, at least in part, in an automated or semiautomated manner using any of the teachings herein.

It is further noted that any disclosure of a device and/or system detailed herein also corresponds to a disclosure of otherwise providing that device and/or system and/or utilizing that device and/or system.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A method, comprising:
obtaining heart phase data for a plurality of activation cycles of a living human afflicted with atrial fibrillation; and
analyzing the heart phase data to identify specific heart tissue locations where there are repeated and consistent temporal discrepancies of electrical activation relative to other tissue locations.

2. The method of claim 1, wherein:
the action of analyzing the heart phase data includes implementing a statistical analysis on the heart phase data.

3. The method of claim 1, wherein:
the action of analyzing the heart phase data includes implementing time averaging analysis on the heart phase data.

4. The method of claim 1, wherein the action of analyzing the heart phase data includes:
for a plurality of spatial locations on an interior surface of the heart, which spatial locations include the identified specific heart tissue locations, identifying respective maximum phase gradients for respective locations of the plurality of spatial locations over a length of time; time averaging the respective maximum phase gradients for the respective locations; and
identifying corresponding locations where the time averaged results are statistically aberrant and/or are not statistically aberrant, wherein
the identified corresponding locations of the time average results that are statistically aberrant are the identified specific heart tissue locations and/or the identified corresponding locations of the time average results that are not statistically aberrant are not the identified specific heart tissue locations.

5. The method of claim 4, wherein:
the respective maximum phase gradients are the respective maximum phase gradients between the respective locations and a plurality of proximate locations on the surface of the heart.

6. The method of claim 5, wherein the proximate locations are effectively North-South-East-West adjacent locations.

7. The method of claim 5, wherein the proximate locations are the locations immediately surrounding the respective location.

8. The method of claim 1, further comprising:
abating a surface of the heart based at least on the identified specific heart tissue locations

9-12. (canceled)

13. The method of claim 1, wherein the action of analyzing the heart phase data includes:
for a plurality of spatial locations on an interior surface of the heart, which spatial locations include the identified specific heart tissue locations, identifying respective maximum phase gradients for respective locations of the plurality of spatial locations over a length of time;
time averaging the respective maximum phase gradients for the respective locations; and
identifying corresponding locations where the time averaged results are non-zero and/or statistically zero, wherein
the identified corresponding locations of the time average results that are non-zero are the identified specific heart tissue locations and/or the identified corresponding locations of the time average results that are statistically zero are not the identified specific heart tissue locations.

14. A method, comprising:
developing a time-varying electrical potential map of a surface of a cavity of a beating heart;
developing a time-varying phase map of the surface of the cavity based on the developed time-varying electrical potential map; and
identifying repeating phase signatures for respective locations on the surface of the atrial cavity from the time-varying phase map that repeat in a statistically aberrant manner relative to other phase signatures at other respective locations.

15. The method of claim 14, wherein:
the electrical potential map has at least 500 electrical potential spatial locations and at least respective 5,000 temporal potential values for the respective potential spatial locations;
the phase map has at least 400 phase spatial locations and at least respective 4,000 temporal phase values for respective phase locations.

16. The method of claim 15, wherein:
respective electrical potential locations of the at least 500 electrical potential locations have respective phase locations of the at least 500 phase locations.

17-18. (canceled)

19. The method of claim 14, wherein:
the action of developing a time-varying electrical potential map of a surface of a cavity of a beating heart is based at least in part on time-varying readings from electrodes located in the cavity; and
the action of identifying the repeating phase signatures is executed within 20 minutes of the electrodes being removed from the chamber.

20. The method of claim 14, wherein:
the action of developing a time-varying electrical potential map of a surface of a cavity of a beating heart is based at least in part on invasive readings taken while a human in which the beating heart resides is in an operating room; and
the action of identifying the repeating phase signatures is executed before the human leaves the operating room.

21. The method of claim 20, further comprising:
executing a medical procedure targeted at tissue of the heart corresponding to at least some of the respective locations identified as having the repeating phase signatures that repeat in the statistically aberrant manner before the human leaves the operating room.

22-23. (canceled)

24. A method, comprising:
developing data including at least X spatial locations and at least Y respective phase gradients for the respective spatial locations of the X spatial locations;
statistically analyzing the developed data; and
identifying locations of the respective locations that are indicative of tissue influencing atrial fibrillation based on the statistical analysis, wherein
X is at least 64 and Y is at least 50.

25. The method of claim 24, wherein:
the statistical analysis is time averaging.

26. (canceled)

27. The method of claim 24, wherein:
the action of identifying locations includes identifying locations where averaging of the maximum phase gradients yields a statistically meaningful non-zero value.

28. The method of claim 24, wherein:
the action of identifying locations includes identifying other locations where averaging of the maximum phase gradients of at least a majority of the Y phase gradients yields a statistically zero value.

29-31. (canceled)

32. The method of claim 24, wherein:
the statistical analysis of the developed data identifies statistically consistent patterns of electrical activity that repeat in a statistically meaningful manner over time.

33-40. (canceled)

41. The method of claim 4, further comprising:
wherein the plurality of spatial locations includes at least 24 locations; and
there are at least 36 temporally spaced respective maximum phase gradients for the respective locations.

42. The method of claim 5, further comprising:
obtaining respective plurality of temporally spaced electrical potentials for respective electrodes of at least 24 electrodes of a catheter located in a heart chamber at a first location in the heart chamber;
converting the obtained respective plurality of temporally spaced electrical potentials to the heart phase data, thereby obtaining the heart phase data, wherein
the actions of identifying respective maximum phase gradients, time averaging, identified corresponding locations where the time average results are statistically aberrant and/or not statistically aberrant, are based on the obtained respective plurality of temporally spaced electrical potentials for the catheter located at the first location.

43. The method of claim 42, further comprising:
obtaining respective second plurality of temporally spaced electrical potentials for respective electrodes of at least 64 electrodes of the catheter located in the heart chamber at a second location in the heart chamber different from the first chamber;
converting the obtained respective second plurality of temporally spaced electrical potentials to second heart phase data;
for a plurality of second spatial locations on the interior surface of the heart, which second spatial locations include the identified specific heart tissue locations, identifying respective second maximum phase gradients for respective second locations of the plurality of second spatial locations over second length of time;
second time averaging the second respective maximum phase gradients for the second respective locations; and
identifying corresponding second locations where the time averaged second results are statistically aberrant and/or not statistical aberrant, wherein
the identified corresponding second locations of the second time average results that are statistically aberrant are included in the identified specific heart tissue locations and/or the identified corresponding second locations of the time second average results that are not statistically aberrant are not included in the identified specific heart tissue locations.

44. The method of claim 43, wherein:
the action of obtaining heart phase data and analyzing is executed in real time vis-à-vis a catheter located in a heart chamber.

45. The method of claim 14, wherein:
the electrical potential map has at least 500 electrical potential locations and at least respective 20,000 temporal locations for respective potential locations of the 500 potential locations;
the phase map has at least 500 phase locations and at least respective 20,000 temporal locations for respective phase locations of the 500 phase locations.

46. The method of claim 14, wherein:
the actions of developing a time-varying electrical potential map, developing the time-varying phase map, and identifying the repeating phase signatures are executed within a period of no more than 20 minutes.

47. The method of claim 21, further comprising:
after executing the medical procedure, developing second time-varying electrical potential map of the surface of the cavity of the beating heart;
developing second time-varying phase map of the surface of the cavity based on the second developed time-varying electrical potential map; and
evaluating whether and/or how many repeating phase signatures for respective locations on the surface of the atrial cavity from the second time-varying phase map that repeat in a statistically aberrant manner relative to other phase signatures at other respective locations, and based on the evaluation, evaluation whether the medical procedure was successful.

48. The method of claim 21, wherein:
the medical procedure is ablation of the targeted tissue.

49. The method of claim 24, wherein:
the action of identifying locations includes identifying locations where the statistical analysis of the developed data indicates non-random activation of respective heart tissue cells at the identified locations.

50. The method of claim 24, wherein:
the action of identifying locations includes identifying locations where averaging of the maximum phase gradients of at least a majority of the Y phase gradients yields a statistically meaningful non-zero value; and
the action of identifying the locations includes further statistically analyzing the values of the non-zero values.

51. The method of claim 24, wherein:
X is at least 300 and Y is at least 75.

52. The method of claim 24, wherein:
X is at least 1,000 and Y is between 60 and 1,000, inclusive.
